SEQUENCE LISTING (72 sequences)

SEQ ID NO: 1 - protein, 36 aa, Artificial Sequence
  Synthetic DPT-PP2A peptides
XXKKKIKREIXXXXXXDTLDHIRALDRLQEVPHEGP

SEQ ID NO: 2 - protein, 16 aa, Artificial Sequence
  Synthetic DPT sequence
XXKKKIKREIXXXXXX

SEQ ID NO: 3 - protein, 20 aa, Artificial Sequence
  Synthetic human PP2A domain interacting with C9
DTLDHIRALDRLQEVPHEGP

SEQ ID NO: 4 - protein, 32 aa, Artificial Sequence
  Synthetic DPT sequence fused to PP2Ah
XXKKKIKREIXXDTLDHIRALDRLQEVPHEGP

SEQ ID NO: 5 - protein, 32 aa, Artificial Sequence
  Synthetic DPT-PP2Ah
VKKKKIKREIKIDTLDHIRALDRLQEVPHEGP

SEQ ID NO: 6 - protein, 34 aa, Artificial Sequence
  Synthetic DPT-C9 peptides
XXKKKIKREIXXXXXXYXETLDGIXEQWAXSXXX

SEQ ID NO: 7 - protein, 30 aa, Artificial Sequence
  Synthetic human DPT-C9
VKKKKIKREIKIYVETLDGIFEQWAHSEDL

SEQ ID NO: 8 - protein, 30 aa, Artificial Sequence
  Synthetic mouse DPT-C9
VKKKKIKREIKIYIETLDGILEQWARSEDL

SEQ ID NO: 9 - protein, 11 aa, Artificial Sequence
  Synthetic DPT sequence
VKKKKIKREIK

SEQ ID NO: 10 - protein, 18 aa, Artificial Sequence
  Synthetic mouse C9 domain interacting with PP2A
YIETLDGILEQWARSEDL

SEQ ID NO: 11 - protein, 30 aa, Artificial Sequence
  Synthetic DPT-C9r
VKKKKIKREIKILDESRAWQELIGDLTEIY

SEQ ID NO: 12 - protein, 11 aa, Artificial Sequence
  Synthetic Tat penetrating peptide
YGRKKRRQRRR

SEQ ID NO: 13 - protein, 12 aa, Artificial Sequence
  Synthetic PP2A peptide
DTLDHIRALDRL

SEQ ID NO: 14 - protein, 12 aa, Artificial Sequence
  Synthetic PP2A peptide
LDHIRALDRLQE

SEQ ID NO: 15 - protein, 12 aa, Artificial Sequence
  Synthetic PP2A peptide
HIRALDRLQEVP

SEQ ID NO: 16 - protein, 12 aa, Artificial Sequence
  Synthetic PP2A peptide
RALDRLQEVPHE

SEQ ID NO: 17 - protein, 12 aa, Artificial Sequence
  Synthetic PP2A peptide
LDRLQEVPHEGP

SEQ ID NO: 18 - protein, 18 aa, Artificial Sequence
  Synthetic Peptide C9h sequence
YVETLDGIFEQWAHSEDL

SEQ ID NO: 19 - protein, 19 aa, Artificial Sequence
  Synthetic PP2Ac peptide
TLDHIRALDRLQEVPHEGP

SEQ ID NO: 20 - protein, 18 aa, Artificial Sequence
  Synthetic PP2Ac peptide
LDHIRALDRLQEVPHEGP

SEQ ID NO: 21 - protein, 17 aa, Artificial Sequence
  Synthetic PP2Ac peptide
DHIRALDRLQEVPHEGP

SEQ ID NO: 22 - protein, 16 aa, Artificial Sequence
  Synthetic PP2Ac peptide
HIRALDRLQEVPHEGP

SEQ ID NO: 23 - protein, 19 aa, Artificial Sequence
  Synthetic PP2Ac peptide
DTLDHIRALDRLQEVPHEG

SEQ ID NO: 24 - protein, 18 aa, Artificial Sequence
  Synthetic PP2Ac peptide
DTLDHIRALDRLQEVPHE

SEQ ID NO: 25 - protein, 17 aa, Artificial Sequence
  Synthetic PP2Ac peptide
DTLDHIRALDRLQEVPH

SEQ ID NO: 26 - protein, 16 aa, Artificial Sequence
  Synthetic PP2Ac peptide
DTLDHIRALDRLQEVP

SEQ ID NO: 27 - protein, 18 aa, Artificial Sequence
  Synthetic PP2Ac peptide
TLDHIRALDRLQEVPHEG

SEQ ID NO: 28 - protein, 16 aa, Artificial Sequence
  Synthetic PP2Ac peptide
LDHIRALDRLQEVPHE

SEQ ID NO: 29 - protein, 17 aa, Artificial Sequence
  Synthetic PP2Ac peptide
TLDHIRALDRLQEVPHE

SEQ ID NO: 30 - protein, 17 aa, Artificial Sequence
  Synthetic PP2Ac peptide
LDHIRALDRLQEVPHEG

SEQ ID NO: 31 - protein, 18 aa, Artificial Sequence
  Synthetic CPP sequence 1
RQKRLIRQKRLIRQKRLI

SEQ ID NO: 32 - protein, 18 aa, Artificial Sequence
  Synthetic CPP sequence 2
RHSRIGRHSRIGRHSRIG

SEQ ID NO: 33 - protein, 16 aa, Artificial Sequence
  Synthetic CPP sequence 3
RHSRIGIIQQRRTRNG

SEQ ID NO: 34 - protein, 16 aa, Artificial Sequence
  Synthetic CPP sequence 4
RHSRIGVTRQRRARNG

SEQ ID NO: 35 - protein, 15 aa, Artificial Sequence
  Synthetic CPP sequence 5
RRRRRRRSRGRRRTY

SEQ ID NO: 36 - protein, 12 aa, Artificial Sequence
  Synthetic CPP sequence 6
XXKKKIKREIXX

SEQ ID NO: 37 - protein, 9 aa, Artificial Sequence
  Synthetic Tat peptide 2
RKKRRQRRR

SEQ ID NO: 38 - protein, 9 aa, Artificial Sequence
  Synthetic Polyarginine peptide R9
RRRRRRRRR

SEQ ID NO: 39 - protein, 11 aa, Artificial Sequence
  Synthetic Polyarginine peptide R11
RRRRRRRRRRR

SEQ ID NO: 40 - protein, 32 aa, Artificial Sequence
  Synthetic HA2-R9 peptide
GLFEAIEGFIENGWEGMIDGWYGRRRRRRRRR

SEQ ID NO: 41 - protein, 16 aa, Artificial Sequence
  Synthetic Penetratin peptide
RQIKIWFQNRRMKWKK

SEQ ID NO: 42 - protein, 27 aa, Artificial Sequence
  Transportan peptide
GWTLNSAGYLLGKINLKALAALAKKIL

SEQ ID NO: 43 - protein, 33 aa, Artificial Sequence
  Synthetic Maurocalcine peptide
GDCLPHLKLCKENKDCCSKKCKRRGTNIEKRCR

SEQ ID NO: 44 - protein, 10 aa, Artificial Sequence
  Synthetic Decalysine peptide
KKKKKKKKKK

SEQ ID NO: 45 - protein, 11 aa, Artificial Sequence
  Synthetic HIV-Tat derived PTD4 peptide
YARAAARQARA

SEQ ID NO: 46 - protein, 12 aa, Artificial Sequence
  Synthetic Hepatitis B virus Translocation Motif (PTM) peptide
PLSSIFSRIGDP

SEQ ID NO: 47 - protein, 28 aa, Artificial Sequence
  Synthetic mPrP1-28 peptide
MANLGYWLLALFVTMWTDVGLCKKRPKP

SEQ ID NO: 48 - protein, 35 aa, Artificial Sequence
  Synthetic POD peptide
GGGARKKAAKAARKKAAKAARKKAAKAARKKAAKA

SEQ ID NO: 49 - protein, 19 aa, Artificial Sequence
  Synthetic pVEC peptide
LLIILRRRRIRKQAHAHSK

SEQ ID NO: 50 - protein, 23 aa, Artificial Sequence
  Synthetic EB1 peptide
LIRLWSHLIHIWFQNRRLKWKKK

SEQ ID NO: 51 - protein, 22 aa, Artificial Sequence
  Synthetic Rath peptide
TPWWRLWTKWHHKRRDLPRKPE

SEQ ID NO: 52 - protein, 20 aa, Artificial Sequence
  Synthetic CADY peptide
GLWRALWRLLRSLWRLLWRA

SEQ ID NO: 53 - protein, 24 aa, Artificial Sequence
  Synthetic Histatin 5 peptide
DSHAKRHHGYKRKFHEKHHSHRGY

SEQ ID NO: 54 - protein, 16 aa, Artificial Sequence
  Synthetic Antp peptide
RQIKIWFQNRRMKWKK

SEQ ID NO: 55 - protein, 16 aa, Artificial Sequence
  Synthetic Cyt86-101 peptide
KKKEERADLIAYLKKA

SEQ ID NO: 56 - protein, 38 aa, Artificial Sequence
  Synthetic PP2Ac chimeric peptide
RQKRLIRQKRLIRQKRLIDTLDHIRALDRLQEVPHEGP

SEQ ID NO: 57 - protein, 38 aa, Artificial Sequence
  Synthetic PP2Ac chimeric peptide
RHSRIGRHSRIGRHSRIGDTLDHIRALDRLQEVPHEGP

SEQ ID NO: 58 - protein, 36 aa, Artificial Sequence
  Synthetic PP2Ac chimeric peptide
RHSRIGIIQQRRTRNGDTLDHIRALDRLQEVPHEGP

SEQ ID NO: 59 - protein, 36 aa, Artificial Sequence
  Synthetic PP2Ac chimeric peptide
RHSRIGVTRQRRARNGDTLDHIRALDRLQEVPHEGP

SEQ ID NO: 60 - protein, 35 aa, Artificial Sequence
  Synthetic PP2Ac chimeric peptide
RRRRRRRSRGRRRTYDTLDHIRALDRLQEVPHEGP

SEQ ID NO: 61 - protein, 34 aa, Artificial Sequence
  Synthetic DPT-PP2A peptide
XXKKKIKREIXXXXXXLDHIRALDRLQEVPHEGP

SEQ ID NO: 62 - protein, 30 aa, Artificial Sequence
  Synthetic DPT-PP2A peptide
XXKKKIKREIXXLDHIRALDRLQEVPHEGP

SEQ ID NO: 63 - protein, 30 aa, Artificial Sequence
  Synthetic DPT-PP2A peptide
VKKKKIKREIKILDHIRALDRLQEVPHEGP

SEQ ID NO: 64 - DNA, 60 bp, Artificial Sequence
  Synthetic PP2Ac nucleotidic sequence
gatacactggatcatatcagagcacttgatcgcctacaagaagttccccatgagggtcca

SEQ ID NO: 65 - DNA, 96 bp, Artificial Sequence
  Synthetic DPT-PP2Ah nucleotidic sequence
gtaaagaagaagaaaatcaagagagagattaagattgatacactggatcatatcagagcacttgatcgcctacaagaagttccccatgagggtcca

SEQ ID NO: 66 - DNA, 114 bp, Artificial Sequence
  Synthetic PP2Ac chimeric peptide nucleotidic sequence
cgccagaaacgcctgattcgccagaaacgcctgattcgccagaaacgcctgattgatacactggatcatatcagagcacttgatcgcctacaagaagttccccatgagggtcca

SEQ ID NO: 67 - DNA, 114 bp, Artificial Sequence
  Synthetic PP2Ac chimeric peptide nucleotidic sequence
cgccatagccgcattggccgccatagccgcattggccgccatagccgcattggcgatacactggatcatatcagagcacttgatcgcctacaagaagttccccatgagggtcca

SEQ ID NO: 68 - DNA, 108 bp, Artificial Sequence
  Synthetic PP2Ac chimeric peptide nucleotidic sequence
cgacatagcagaataggcattattcaacagaggagaacaagaaatggagatacactggatcatatcagagcacttgatcgcctacaagaagttccccatgagggtcca

SEQ ID NO: 69 - DNA, 108 bp, Artificial Sequence
  Synthetic PP2Ac chimeric peptide nucleotidic sequence
cgccatagccgcattggcgtgacccgccagcgccgcgcgcgcaacggcgatacactggatcatatcagagcacttgatcgcctacaagaagttccccatgagggtcca

SEQ ID NO: 70 - DNA, 105 bp, Artificial Sequence
  Synthetic PP2Ac chimeric peptide nucleotidic sequence
cgccgccgccgccgccgccgcagccgcggccgccgccgcacctatgatacactggatcatatcagagcacttgatcgcctacaagaagttccccatgagggtcca

SEQ ID NO: 71 - DNA, 90 bp, Artificial Sequence
  Synthetic PP2Ac chimeric peptide nucleotidic sequence
gtaaagaagaagaaaatcaagagagagattaagattctggatcatatcagagcacttgatcgcctacaagaagttccccatgagggtcca

SEQ ID NO: 72 - protein, 18 aa, Artificial Sequence
  Synthetic C9x peptide
YXETLDGIXEQWAXSXXX

(19) United States
(12) Patent Application Publication
(10) Pub. No.: US 2013/0303439 A1
(43) Pub. Date: Nov. 14, 2013

(54) CHIMERIC PEPTIDES INCLUDING A PENETRATING PEPTIDE AND A BINDING DOMAIN OF PP2A CATALYTIC SUBUNIT TO CASPASE-9

(71) Applicants: Angelita Rebollo Garcia, Paris (FR); Didier Decaudin, Paris (FR); Fariba Nemati, Paris (FR)

(72) Inventors: Angelita Rebollo Garcia, Paris (FR); Didier Decaudin, Paris (FR); Fariba Nemati, Paris (FR)

(73) Assignees: Institut National de la Sante et de la Recherche Medicale (INSERM), Paris (FR); INSTITUT CURIE, Paris cedex 05 (FR)

(21) Appl. No.: 13/876,604

(22) PCT Filed: Sep. 30, 2011
(86) PCT No.: PCT/EP11/67173

§ 371 Date: Aug. 1, 2013

(30) Foreign Application Priority Data
Sep. 30, 2010 (EP) 10306059.6

(51) Int. Cl. C12N 9/16; G01N 33/573; A61K 38/46; A61K 45/06; C07K 7/06; C07K 7/08

(52) U.S. Cl. CPC C12N 9/16; C07K 7/06; C07K 7/08; A61K 38/465; A61K 45/06; G01N 33/573
USPC 514/4.4; 530/326; 530/300; 530/324; 536/23.5; 435/69.1; 514/18.9; 435/7.4; 435/69.7

(57) ABSTRACT

The invention related to chimeric peptides including a penetrating peptide and a binding domain of PP2A catalytic subunit to caspase-9 which have pro-apoptotic activity. These chimeric peptides may be used for the treatment of hyperproliferative disorders.

The invention related to chimeric peptides including a penetrating peptide and a binding domain of PP2A catalytic subunit to caspase-9 which have pro-apoptotic activity. These chimeric peptides may be used for the treatment of hyperproliferative disorders.

Apoptosis is a regulated process important for differentiation, control of cell number and removal of damaged cells. Failure to regulate apoptosis is a common feature in several diseases, including autoimmune disorders, neurodegenerative diseases and cancer. Apoptosis occurs through the activation of a cell-intrinsic suicide programme and is carried out by internal as well as external signals. The process of apoptosis can be divided in various phases that, at the end, activate signals leading to cell destruction.

Although apoptosis is induced by a wide range of death stimuli, the execution phase of apoptosis is carried out, among others, by the caspases, that cleave target proteins leading to cell morphological changes.

Phosphorylation plays a critical role in the regulation of cell physiology and dysregulation of the mechanisms contributes to many disease states. Although much is known regarding alterations in kinase function in diseases such as cancer, the role of specific phosphatases in these same processes remains less characterized.

Serine/threonine phosphatases are usually classified as type 1 (PP1) or type 2 (PP2), depending on their substrate specificity and sensitivity to inhibitors.

PP1 represents a family of holoenzymes generated by specific interactions between catalytic subunits and a wide variety of regulatory or targeting subunits. PP1 is a major eukaryotic phosphatase that regulates diverse cellular processes such as cell cycle progression, proliferation, protein synthesis, muscle contraction, carbohydrate metabolism, transcription, cytokinesis and neuronal signalling. During cell cycle, PP1 activity is regulated by phosphorylation. PP1 plays a key role in the mitotic transition by dephosphorylating proteins that are essential in these cellular functions. It has been shown that phosphorylation of PP1a at threonine 320 by cyclin-dependent kinases inhibits its enzymatic activity. In agreement, a constitutive mutant of PP1a that is resistant to cdk phosphorylation prevents cells from entering the S phase of cell cycle. Furthermore it was shown that IL-2 deprivation-induced apoptosis operates by regulating Bad dephosphorylation through the PP1a phosphatise (Ayllon et al., 2000, EMBO J. 19: 1-10) and that PP1a associates to caspase-9 to induce its dephosphorylation and, as a consequence, its protease activity (Dessauge et al., 2006. J. Immunol. 177, 2441-2451).

Serine/threonine protein phosphatase 2A (PP2A) refers to a large family of dimeric or trimeric enzymes. The PP2A core enzyme consists of a catalytic C subunit (PP2Ac) and a structural A subunit. A third subunit (B) eventually binds to the core and these B subunits regulate both the substrate specificity and localization of PP2A holoenzymes. The A subunit primary serves a structural role and single amino acid alterations disrupt the binding of specific B subunits, suggesting that the A subunit regulates PP2A holoenzyme composition. Various PP2A complexes have been implicated in the control of a variety of cellular processes, including cell proliferation, survival, adhesion, cytoskeletal dynamics and malignant transformation.

A role of PP2A in apoptosis is suggested by its interaction with caspase-3, Bcl-2 and adenovirus E4orf4 protein. The activity of Bcl-2, an anti-apoptotic protein, is regulated by phosphorylation on Ser70, which is required for its anti-apoptotic role and can be reversed by PP2A. Moreover, IL-3 or bryostatin-1-induced phosphorylation of Bcl-2 on Ser70 is followed by increased association between Bcl-2 and PP2A prior to dephosphorylation of Bcl-2. Finally, PP2A regulates apoptosis via modulation of apoptotic signals such as NFkB, ERK and PI3K signalling pathways.

Penetrating peptides interacting with PP1/PP2A proteins were designed. This approach, named “Drug Phosphatase Technology” (DPT), was described in Guergnon et al. (Mol. Pharmacol. (2006) 69:1115-1124) and in the International patent applications WO2003/011898 and WO2004/011595.

The inventors have now shown that PP2A interacts with caspase-9. The inventors found that a particular sequence from the C-terminal portion of caspase-9 protein is a PP2Ac-binding domain. This sequence was identified as being YIETLDGILEQWARSEDL (SEQ ID NO:10) for murine caspase-9, and as being YVETLDGIFEQWAHSEDL (SEQ ID NO:18) for human caspase-9. This binding domain to PP2Ac corresponds to amino acid positions 401-418 of murine caspase-9 (NCBI accession number NP_{—}056548), amino acid positions 363-380 of human caspase-9 (NCBI accession number NP_{—}001220).

The inventors further demonstrated that this caspase-9 PPA2c-binding domain, when fused to a penetrating peptide which interacts with PP1-PP2A (fusion peptide DPT-C9 and DPT-C9h), becomes a therapeutic molecule able to deregulate survival of human cells.

The inventors have additionally identified the corresponding sequence from the human PP2Ac subunit which interacts with its partner caspase-9 (DTLDHIRALDRLQEVPHEGP, SEQ ID NO:3, positions 175-194 of human PPA2c sequence deposited in Swiss-Prot database under accession number P67775-1). This binding domain to caspase-9 corresponds to amino acid positions. This PP2Ac caspase-9-binding domain, when fused to a penetrating peptide which interacts with PP1-PP2A (fusion peptide DPT-PP2Ah), also becomes a therapeutic molecule able to induce cell apoptosis, even with a better efficacy than its counterpart the fusion peptide comprising human caspase-9 PP2Ac-binding domain (DPT-C9h).

### SUMMARY OF THE INVENTION

The invention provides an isolated PP2Ac peptide comprising or consisting of:

a) the amino acid sequence DTLDHIRALDRLQEVPHEGP (SEQ ID NO:3);

b) an amino acid sequence substantially homologous to SEQ ID NO:3, preferably at least 80% identical to SEQ ID NO:3, which induces cell apopotosis; or

c) a proteolysis-resistant peptide which induces cell apoptosis and which derives from the peptide defined in a) or b) by one or more chemical modifications.

In a preferred embodiment, the peptide comprises or consists of the sequence DTLDHIRALDRLQEVPHEGP (SEQ ID NO: 3).

In some embodiments, the PP2Ac peptide is linked with at least one cell penetrating peptide, forming a PP2Ac chimeric peptide.

In another aspect, the invention relates to an isolated “DPT-PP2A” peptide, i.e. a peptide comprising, or consisting of:

a) the amino acid sequence X1-KKKIKREI-X2-X3-DTLDHIRALDRLQEVPHEGP (SEQ ID NO:1)

wherein X1 is vacant, is a lysine residue, or is valine-lysine;

X2 is vacant, is a lysine residue, or is lysine-isoleucine;

X3 is vacant or is an amino acid sequence of 1 to 4 amino acids;

b) an amino acid sequence substantially homologous to SEQ ID NO:1, preferably at least 80% identical to SEQ ID NO:1, which induces cell apoptosis; or

c) a proteolysis-resistant peptide which induces cell apoptosis and which derives from the peptide defined in a) or b) by one or more chemical modifications.

Also encompasses are polynucleotides comprising or consisting of a nucleotidic sequence encoding a peptide according to the invention, polynucleotides with nucleotidic sequences complementary to one of the above sequences and sequences hybridizing to said polynucleotides under stringent conditions.

The invention further relates to a genetic construct consisting of or comprising a polynucleotide as defined herein, and regulatory sequences (such as a suitable promoter(s), enhancer(s), terminator(s), etc.) allowing the expression (e.g. transcription and translation) of a peptide according to the invention in a host cell.

Thus, in another aspect, the invention relates to a host or host cell that expresses (or that under suitable circumstances is capable of expressing) a peptide of the invention; and/or that contains a polynucleotide of the invention or genetic construct of the invention.

The invention further relates to methods for preparing or generating the peptides of the invention.

The invention further relates to a pharmaceutical composition comprising a peptide of the invention, together with a pharmaceutically acceptable carrier, and to the use of the peptides or the pharmaceutical composition according to the invention for treating hyperproliferative diseases or parasitic diseases.

### DEFINITIONS

The terms “cell penetrating peptide” or “CPP” are used interchangeably and refer to cationic cell penetrating peptides, also called transport peptides, carrier peptides, or peptide transduction domains. The CPP, as shown herein, have the capability of inducing cell penetration of a peptide fused to the CPP within 30%, 40%, 50%, 60%, 70%, 80%, 90%, or 100% of cells of a given cell culture population, including all integers in between, and allow macromolecular translocation within multiple tissues in vivo upon systemic administration. A cell-penetrating peptide may also refers to a peptide which, when brought into contact with a cell under appropriate conditions, passes from the external environment in the intracellular environment, including the cytoplasm, organelles such as mitochondria, or the nucleus of the cell, in conditions significantly greater than passive diffusion. Such penetrating peptides may be those described in Fonseca S. B. et al., Advanced Drug Delivery Reviews, 2009, 61: 953-964, Johansson et al., Methods in Molecular Biology, 2011, Vol. 683, Chapter 17, in WO2004/011595 and in WO2003/011898.

A peptide “substantially homologous” to a reference peptide may derive from the reference sequence by one or more conservative substitutions. Preferably, these homologous peptides do not include two cysteine residues, so that cyclization is prevented. Two amino acid sequences are “substantially homologous” or “substantially similar” when one or more amino acid residue are replaced by a biologically similar residue or when greater than 80% of the amino acids are identical, or greater than about 90%, preferably greater than about 95%, are similar (functionally identical). Preferably, the similar, identical or homologous sequences are identified by alignment using, for example, the GCG (Genetics Computer Group, Program Manual for the GCG Package, Version 7, Madison, Wis.) pileup program, or any of the programs known in the art (BLAST, FASTA, etc.). The percentage of identity may be calculated by performing a pairwise global alignment based on the Needleman-Wunsch alignment algorithm to find the optimum alignment (including gaps) of two sequences along their entire length, for instance using Needle, and using the BLOSUM62 matrix with a gap opening penalty of 10 and a gap extension penalty of 0.5.

The term “conservative substitution” as used herein denotes the replacement of an amino acid residue by another, without altering the overall conformation and function of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Neutral hydrophilic amino acids, which can be substituted for one another, include asparagine, glutamine, serine and threonine.

By “substituted” or “modified” the present invention includes those amino acids that have been altered or modified from naturally occurring amino acids.

As such, it should be understood that in the context of the present invention, a conservative substitution is recognized in the art as a substitution of one amino acid for another amino acid that has similar properties.

Examples of conservative substitutions are set out in the Table 1 below:

TABLE 1
Conservative substitutions I

| Side-chain characteristic | Amino acid |
|---|---|
| Non-polar | G A P I L V |
| Polar-uncharged | C S T M N Q |
| Polar-charged | D E K R |
| Aromatic | H F W Y |
| Other | N Q D E |

Alternatively, conservative amino acids can be grouped as described in Lehninger (1975, Biochemistry, Second Edition, Worth Publishers, Inc. New-York: NY., pp. 71-77), as set out in Table 2, immediately below.

TABLE 2
conservative substitutions II

| Side-chain characteristic | | Amino acid |
|---|---|---|
| Non-polar | Aliphatic | A L I V P |
| | Aromatic | F W |
| | Sulphur-containing | M |
| | Borderline | G |
| Uncharged-polar | Hydroxyl | S T Y |
| | Amides | N Q |
| | Sulfhydryl | C |
| | Borderline | G |
| Positively charged (basic) | | K R H |
| Negatively charged (acidic) | | D E |

As still another alternative, exemplary conservative substitutions are set out in Table 3, immediately below.

TABLE 3
conservative substitutions III

| Original residue | Exemplary susbtitution |
|---|---|
| A | V L I |
| R | K Q N |
| N | Q H K R |
| D | E |
| C | S |
| G | N |
| E | D |
| H | N Q K R |
| I | L V M A F |
| L | I V M A F |
| K | R Q N |
| M | L F I |
| F | L V I A |
| P | G |
| S | T |
| T | S |
| W | Y |
| Y | W F T S |
| V | I L M F A |

The N- and C-termini of the peptides described herein may be protected against proteolysis. For instance, the N-terminus may be in the form of an acetyl group, and/or the C-terminus may be in the form of an amide group. Internal modifications of the peptides to be resistant to proteolysis are also envisioned, e.g. wherein at least a —CONH— peptide bond is modified and replaced by a (CH2NH) reduced bond, a (NHCO) retro-inverso bond, a (CH2-O) methylene-oxy bond, a (CH2-S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH-bond. The peptides described herein may also be protected against proteolysis by the technique of stapled peptides as described by Walensky et al. Science. 2004, 305, 1466-70.

In another aspect of the invention, peptides are covalently bound to a polyethylene glycol (PEG) molecule by their C-terminal terminus or a lysine residue, notably a PEG of 1500 or 4000 MW, for a decrease in urinary clearance and in therapeutic doses used and for an increase of the half-life in blood plasma. In yet another embodiment, peptide half-life is increased by including the peptide in a biodegradable and biocompatible polymer material for drug delivery system forming microspheres. Polymers and copolymers are, for instance, poly(D, L-lactide-co-glycol ide) (PLGA) (as illustrated in US2007/0184015, SoonKap Hahn et al).

A peptide according to the invention may have a length comprised between 16 to 70 amino acids, preferably between 20 to 40 amino acids. Still more preferably a peptide according to the invention may have a length of 16, 17, 18, 19, 20, 25, 30, 35, 40, 45, 50, 55, 60, 65 or 70 amino acids.

By “stringent conditions”, it is meant conditions of temperature and ionic strength allowing specific hybridization between two complementary nucleic acid fragments and limiting non-specific binding (Sambrook et al. Molecular Cloning, Second Edition (1989), 9.47-9.62). The temperature conditions are generally comprised between (Tm−5° C.) and (Tm−10° C.), Tm being the theoretical fusion temperature, which is defined as the temperature at which 50% of the paired strands separate. For sequences comprising more than 30 bases, Tm is defined by the formula: Tm=81.5+0.41 (% G+C)+16.6 Log (cations concentration)−0.63 (% formamide)−(600/bases number). For sequences comprising less than 30 bases, Tm is defined by the formula: Tm=4 (G+C)+2 (A+T).

As used herein, the term “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The term “patient” or “subject” refers to a human or non human mammal, preferably a mouse, cat, dog, monkey, horse, cattle (i.e. cow, sheep, goat, buffalo), including male, female, adults and children.

As used herein, the term “treatment” or “therapy” includes curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of: halting the onset, reducing the risk of development, reducing the incidence, delaying the onset, reducing the development, as well as increasing the time to onset of symptoms of a particular disorder.

As used herein, the term “hyperproliferative diseases” denotes a disease associated with cells which have hyperproliferative capacity, which is either constitutive (activated cells which are continuously cycling) or inducible (resting cells triggered into hyperproliferation by exposure to a cytokine or growth factor).

As used herein, the term “parasitic diseases” denotes an infectious disease caused or transmitted by a parasite.

PP2Ah Peptides

In one aspect, the invention provides an isolated “PP2Ac” peptide, i.e. a peptide comprising or consisting of:

a) the amino acid sequence DTLDHIRALDRLQEVPHEGP (SEQ ID NO: 3);

b) an amino acid sequence substantially homologous to SEQ ID NO: 3, preferably at least 80% identical to SEQ ID NO: 3, which induces cell apoptosis; or

c) a proteolysis-resistant peptide which induces cell apoptosis and which derives from the peptide defined in a) or b) by one or more chemical modifications.

Preferably, a PP2Ac peptide according to the invention induces cell apoptosis, in vitro and/or in vivo. Preferably, the PP2Ac peptide induces apoptosis in Daudi cells as well as in primary cells isolated from xenograft models of lung cancer, uveal melanoma and breast cancer.

Assays for determining if a molecule, for instance a peptide, induces cell apoptosis are well-known in the art and include, for instance, incubating cells with the candidate peptide and determining if apoptosis is induced by said candidate peptide, e.g. by Annexin V and PI labelling of cells and identifying as apoptotic cells, those being Annexin V⁺ and PI⁻ as described in the “Cell death assay” of example 1.

Preferably, the PP2Ac peptide comprises or consists of a sequence at least 80% identical to SEQ ID NO: 3, or a proteolysis-resistant peptide derived therefrom by one or more chemical modifications. The sequence at least 80% identical to SEQ ID NO: 3 may be in particular a natural variant of amino acid positions 175-194 of human PP2Ac (Swiss-Prot P67775-1), or the sequence corresponding to amino acid positions 175-194 of human PP2Ac in a mammalian counterpart (e.g. mouse, rat, monkey, cat, dog, horse) sequence of PP2Ac.

In particular, the PP2Ac peptide comprising or consisting of a sequence at least 80% identical to SEQ ID NO: 3 may have a deletion of one, two, three or four amino acids of the N-terminal or C-terminal part of SEQ ID NO: 3, for example, said PP2Ac peptide may be TLDHIRALDRLQEVPHEGP (SEQ ID NO: 19), LDHIRALDRLQEVPHEGP (SEQ ID NO: 20), DHIRALDRLQEVPHEGP (SEQ ID NO: 21), HIRALDRLQEVPHEGP (SEQ ID NO: 22), DTLDHIRALDRLQEVPHEG (SEQ ID NO: 23), DTLDHIRALDRLQEVPHE (SEQ ID NO: 24), DTLDHIRALDRLQEVPH (SEQ ID NO: 25), or DTLDHIRALDRLQEVP (SEQ ID NO: 26). The PP2Ac peptide comprising or consisting of a sequence at least 80% identical to SEQ ID NO: 3 may also have a deletion of one or two amino acids on the C-terminal part of SEQ ID NO:3 and a deletion of one or two amino acids of the N-terminal part of SEQ ID NO:3, for example said PP2Ac peptide may have the sequence TLDHIRALDRLQEVPHEG (SEQ ID NO: 27), LDHIRALDRLQEVPHE (SEQ ID NO: 28), TLDHIRALDRLQEVPHE (SEQ ID NO: 29), or LDHIRALDRLQEVPHEG (SEQ ID NO: 30).

In a particularly preferred embodiment, the PP2Ac peptide comprising or consisting of a sequence at least 80% identical to SEQ ID NO: 3 is LDHIRALDRLQEVPHEGP (SEQ ID NO: 20).

Preferably, the PP2Ac peptide comprises or consists of the sequence DTLDHIRALDRLQEVPHEGP (SEQ ID NO: 3).

Chimeric Peptides

In some embodiments, the PP2Ac peptide is linked with at least one cell penetrating peptide, forming a PP2Ac chimeric peptide.

In a preferred embodiment, the cell penetrating peptide comprises or consists of:

a) X1-KKKIKREI-X2-X3 (SEQ ID NO: 2)

wherein X1 is vacant, is a lysine residue, or is valine-lysine;

X2 is vacant, is a lysine residue, or is lysine-isoleucine;

X3 is vacant or is an amino acid sequence of 1 to 4 amino acids;

b) (RQKRLI)₃ (SEQ ID NO: 31), (RHSRIG)₃ (SEQ ID NO: 32), RHSRIGIIQQRRTRNG (SEQ ID NO: 33), RHSRIGVTRQRRARNG (SEQ ID NO: 34), RRRRRRRSRGRRRTY (SEQ ID NO: 35),

c) an amino acid sequence homologous to a) or b), or

d) Tat peptide, polyarginines peptide, HA2-R₉ peptide, Penetratin peptide, Transportan peptide, Vectocell® peptide, maurocalcine peptide, decalysine peptide, HIV-Tat derived PTD4 peptide, Hepatitis B virus Translocation Motif (PTM) peptide, mPrP₁₋₂₈ peptide, POD, pVEC, EB1, Rath, CADY, Histatin 5, Antp peptide, Cyt⁸⁶⁻¹⁰¹ peptide.

In an embodiment, in the cell penetrating peptide of a), X3 is vacant, i.e. the cell penetrating peptide is X1-KKKRIKREI-X2 (SEQ ID NO: 36).

In another embodiment, in the cell penetrating peptide of a), X1 is VK, X2 is KI and X3 is vacant, i.e. the cell penetrating peptide is VKKKKIKREIKI (SEQ ID NO: 9).

By “Tat peptide”, it is meant a peptide having the sequence RKKRRQRRR (SEQ ID NO: 37, Tat peptide 2) or YGRKKRRQRRR, (SEQ ID NO: 12).

By “polyarginines peptide”, it is meant a peptide consisting of at least 9 arginines. Preferably, a polyarginine peptide is a peptide having the sequence R₉ (SEQ ID NO: 38) or R₁₁ (SEQ ID NO: 39).

By “HA2-R₉ peptide”, it is meant a peptide having the sequence GLFEAIEGFIENGWEGMIDGWYG-R₉ (SEQ ID NO: 40).

By “Penetratin peptide”, it is meant a peptide having the sequence RQIKIWFQNRRMKWKK (SEQ ID NO: 41).

By “Transportan peptide”, it is meant a peptide having the sequence GWTLNSAGYLLGKINLKALAALAKKIL (SEQ ID NO: 42).

By “Vectocell® peptide”, it is meant a peptide originating from human heparin binding proteins and/or anti-DNA antibodies.

By “Maurocalcine peptide”, it is meant a peptide having the sequence GDCLPHLKLCKENKDCCSKKCKRRGTNIEKRCR (SEQ ID NO: 43).

By “decalysine peptide”, it is meant a peptide having the sequence KKKKKKKKKK (K₁₀) (SEQ ID NO: 44).

By “HIV-Tat derived PTD4 peptide”, it is meant a peptide having the sequence YARAAARQARA (SEQ ID NO: 45).

By “Hepatitis B virus Translocation Motif (PTM) peptide”, it is meant a peptide having the sequence PLSSIFSRIGDP (SEQ ID NO: 46).

By “mPrP₁₋₂₈ peptide”, it is meant a peptide having the sequence MANLGYWLLALFVTMWTDVGLCKKRPKP (SEQ ID NO: 47).

By “POD peptide”, it is meant a peptide having the sequence GGG(ARKKAAKA)₄ (SEQ ID NO: 48).

By “pVEC peptide”, it is meant a peptide having the sequence LLIILRRRRIRKQAHAHSK (SEQ ID NO: 49).

By “EB1 peptide”, it is meant a peptide having the sequence LIRLWSHLIHIWFQNRRLKWKKK (SEQ ID NO: 50).

By “Rath peptide”, it is meant a peptide having the sequence TPWWRLWTKWHHKRRDLPRKPE (SEQ ID NO: 51).

By “CADY peptide”, it is meant a peptide having the sequence GLWRALWRLLRSLWRLLWRA (SEQ ID NO: 52).

By “Histatin 5 peptide”, it is meant a peptide having the sequence DSHAKRHHGYKRKFHEKHHSHRGY (SEQ ID NO: 53).

By “Antp peptide”, it is meant a peptide having the sequence RQIKIWFQNRRMKWKK (SEQ ID NO: 54).

By “Cyt⁸⁶⁻¹⁰¹ peptide”, it is meant a peptide having the sequence KKKEERADLIAYLKKA (SEQ ID NO: 55).

In another preferred embodiment, the PP2Ac peptide is linked to two, three or more penetrating peptides.

The invention further provides an isolated peptide comprising or consisting of:
a) the amino acid sequence (RQKRLI)₃-DTLDHIRALDRLQEVPHEGP (SEQ ID NO:56),
b) the amino acid sequence (RHSRIG)₃-DTLDHIRALDRLQEVPHEGP (SEQ ID NO:57),
c) the amino acid sequence RHSRIGIIQQRRTRNG-DTLDHIRALDRLQEVPHEGP (SEQ ID NO:58),
d) the amino acid sequence RHSRIGVTRQRRARNG-DTLDHIRALDRLQEVPHEGP (SEQ ID NO:59),
e) the amino acid sequence RRRRRRRSRGRRRTY-DTLDHIRALDRLQEVPHEGP (SEQ ID NO:60),
f) an amino acid sequence substantially homologous to a peptide defined in a) to e), preferably at least 80% identical to a peptide defined in a) to e), which induces cell apoptosis;
g) a proteolysis-resistant peptide which induces cell apoptosis and which derives from the peptide defined in a) to f) by one or more chemical modifications.

A PP2Ac chimeric peptide according to the invention induces cell apoptosis, in vitro and/or in vivo. In particular, the PP2Ac chimeric peptide induces apoptosis in Daudi cells as well as in primary cells isolated from xenograft models of lung cancer, uveal melanoma and breast cancer.

Preferably, the PP2Ac chimeric peptide comprises or consists of the sequence SEQ ID NO: 31, SEQ ID NO: 32, SEQ ID NO: 33, SEQ ID NO: 34 or SEQ ID NO: 35 fused to a sequence at least 80% identical to DTLDHIRALDRLQEVPHEGP (SEQ ID NO: 3), or a proteolysis-resistant peptide derived therefrom by one or more chemical modifications. The sequence at least 80% identical to SEQ ID NO: 3 may be in particular a natural variant of amino acid positions 175-194 of human PP2Ac (Swiss-Prot P67775-1), or the sequence corresponding to amino acid positions 175-194 of human PP2Ac in a mammalian counterpart (e.g. mouse, rat, monkey, cat, dog, horse) sequence of PP2Ac. Preferably, the sequence at least 80% identical to SEQ ID NO: 3 may be TLDHIRALDRLQEVPHEGP (SEQ ID NO: 19), LDHIRALDRLQEVPHEGP (SEQ ID NO: 20), DHIRALDRLQEVPHEGP (SEQ ID NO: 21), HIRALDRLQEVPHEGP (SEQ ID NO: 22), DTLDHIRALDRLQEVPHEG (SEQ ID NO: 23), DTLDHIRALDRLQEVPHE (SEQ ID NO: 24), DTLDHIRALDRLQEVPH (SEQ ID NO: 25), DTLDHIRALDRLQEVP (SEQ ID NO: 26), TLDHIRALDRLQEVPHEG (SEQ ID NO: 27), LDHIRALDRLQEVPHE (SEQ ID NO: 28), TLDHIRALDRLQEVPHE (SEQ ID NO: 29), or LDHIRALDRLQEVPHEG (SEQ ID NO: 30).

In particular, the invention relates to an isolated “DPT-PP2A” peptide, i.e. a peptide comprising, or consisting of:

a) the amino acid sequence X1-KKKIKREI-X2-X3-DTLDHIRALDRLQEVPHEGP (SEQ ID NO:1)

wherein X1 is vacant, is a lysine residue, or is valine-lysine;

X2 is vacant, is a lysine residue, or is lysine-isoleucine;

X3 is vacant or is an amino acid sequence of 1 to 4 amino acids;

b) an amino acid sequence substantially homologous to SEQ ID NO:1, preferably at least 80% identical to SEQ ID NO:1, which induces cell apoptosis; or

c) a proteolysis-resistant peptide which induces cell apoptosis and which derives from the peptide defined in a) or b) by one or more chemical modifications.

A DPT-PP2A peptide according to the invention induces cell apoptosis, in vitro and/or in vivo. In particular, the DPT-PP2A peptide induces apoptosis in Daudi cells as well as in primary cells isolated from xenograft models of lung cancer, uveal melanoma and breast cancer.

Preferably, the DPT-PP2A peptide comprises or consists of the sequence X1-KKKIKREI-X2-X3 (SEQ ID NO:2) fused to a sequence at least 80% identical to DTLDHIRALDRLQEVPHEGP (SEQ ID NO:3), or a proteolysis-resistant peptide derived therefrom by one or more chemical modifications. The sequence at least 80% identical to SEQ ID NO:3 may be in particular a natural variant of amino acid positions 175-194 of human PP2Ac (Swiss-Prot P67775-1), or the sequence corresponding to amino acid positions 175-194 of human PP2Ac in a mammalian counterpart (e.g. mouse, rat, monkey, cat, dog, horse) sequence of PP2Ac. Preferably, the sequence at least 80% identical to SEQ ID NO: 3 may be TLDHIRALDRLQEVPHEGP (SEQ ID NO: 19), LDHIRALDRLQEVPHEGP (SEQ ID NO: 20), DHIRALDRLQEVPHEGP (SEQ ID NO: 21), HIRALDRLQEVPHEGP (SEQ ID NO: 22), DTLDHIRALDRLQEVPHEG (SEQ ID NO: 23), DTLDHIRALDRLQEVPHE (SEQ ID NO: 24), DTLDHIRALDRLQEVPH (SEQ ID NO: 25), DTLDHIRALDRLQEVP (SEQ ID NO: 26), TLDHIRALDRLQEVPHEG (SEQ ID NO: 27), LDHIRALDRLQEVPHE (SEQ ID NO: 28), TLDHIRALDRLQEVPHE (SEQ ID NO: 29), or LDHIRALDRLQEVPHEG (SEQ ID NO: 30).

In a particular preferred embodiment, the sequence at least 80% identical to SEQ ID NO: 3 is LDHIRALDRLQEVPHEGP (SEQ ID NO: 20).

Accordingly, in a particular preferred embodiment, the DPT-PP2A peptide comprises or consists of the amino acid sequence X1-KKKIKREI-X2-X3-LDHIRALDRLQEVPHEGP (SEQ ID NO: 61).

According to an embodiment, in the DPT-PP2A peptide of the invention X3 is vacant, i.e. the peptide comprises or consists of:

a) the sequence X1-KKKIKREI-X2-DTLDHIRALDRLQEVPHEGP (SEQ ID NO:4) wherein X1 and X2 are as defined above;

b) amino acid sequence at least 80% identical to SEQ ID NO:4; or

c) a proteolysis-resistant peptide deriving the peptide defined in a) or b) by one or more chemical modifications.

According to a preferred embodiment, X1 is VK, X2 is KI and X3 is vacant, i.e. the DPT-PP2A peptide of the invention comprises or consists of:

a) the sequence VKKKKIKREIKIDTLDHIRALDRLQEVPHEGP (SEQ ID NO:5), also designated DPT-PP2Ah;

b) amino acid sequence at least 80% identical to SEQ ID NO:5; or

c) a proteolysis-resistant peptide deriving the peptide defined in a) or b) by one or more chemical modifications.

In particular, the DPT-PP2A peptide of the invention may comprise or consist of a sequence:

(i) at least 85%, 90%, 95%, or 97% identical to SEQ ID NO:1, SEQ ID NO:4 or SEQ ID NO:5,

(ii) X1-KKKIKREI-X2-X3 (SEQ ID NO:2) fused to a sequence at least 85%, 90%, 95%, or 97% identical to DTLDHIRALDRLQEVPHEGP (SEQ ID NO:3); or

(iii) a proteolysis-resistant peptide deriving the peptide defined in (i) or (ii) by one or more chemical modifications.

Preferably, in the sequence SEQ ID NO: 3, the amino acids shown in bold are unmodified: DTLDHIRALDRLQEVPHEGP.

Preferably, the sequence at least 85% identical to DTLDHIRALDRLQEVPHEGP (SEQ ID NO:3) may be LDHIRALDRLQEVPHEGP (SEQ ID NO:19).

Thus, according to another embodiment, in the DPT-PP2A peptide of the invention X3 is vacant, and the peptide comprises or consists of:

a) the sequence X1-KKKIKREI-X2-LDHIRALDRLQEVPHEGP (SEQ ID NO:62) wherein X1 and X2 are as defined above;

b) amino acid sequence at least 80% identical to SEQ ID NO:62; or

c) a proteolysis-resistant peptide deriving the peptide defined in a) or b) by one or more chemical modifications.

According to another preferred embodiment, X1 is VK, X2 is KI and X3 is vacant, and the DPT-PP2A peptide of the invention comprises or consists of:

a) the sequence VKKKKIKREIKILDHIRALDRLQEVPHEGP (SEQ ID NO:63), also designated DPT-PP2Ah;

b) amino acid sequence at least 80% identical to SEQ ID NO:63; or

c) a proteolysis-resistant peptide deriving the peptide defined in a) or b) by one or more chemical modifications.

The sequence at least 80% identical to SEQ ID NO:1, SEQ ID NO:3, SEQ ID NO:4 SEQ ID NO:5, SEQ ID NO: 56, SEQ ID NO: 57, SEQ ID NO: 58, SEQ ID NO: 59, SEQ ID NO: 60, SEQ ID NO:62, or SEQ ID NO:63 may differ from the reference sequence (i.e. SEQ ID NO:1, SEQ ID NO:3, SEQ ID NO:4 or SEQ ID NO:5, SEQ ID NO: 56, SEQ ID NO: 57, SEQ ID NO: 58, SEQ ID NO: 59, SEQ ID NO: 60, SEQ ID NO:62, or SEQ ID NO:63) by one or more substitution conservative modifications, preferably only by substitution conservative modification(s).

Nucleic Acids

The invention also relates to a polynucleotide comprising or consisting of a nucleotidic sequence encoding a peptide according to the invention. In an embodiment, the polynucleotide comprises or consists of a nucleotidic sequence selected from:

SEQ ID NO: 64
(5′-GATACACTGGATCATATCAGAGCACTTGATCGCCTACAAGAAGTTC
CCCATGAGGGTCCA-3′);
SEQ ID NO: 65
(5′-GTAAAGAAGAAGAAAATCAAGAGAGAGATTAAGATTGATACACTGG
ATCATATCAGAGCACTTGATCGCCTACAAGAAGTTCCCCATGAGGGTCC
A-3′);
SEQ ID NO: 66
(5′-CGCCAGAAACGCCTGATTCGCCAGAAACGCCTGATTCGCCAGAAAC
GCCTGATTGATACACTGGATCATATCAGAGCACTTGATCGCCTACAAGAA
GTTCCCCATGAGGGTCCA-3′);
SEQ ID NO: 67
(5′-CGCCATAGCCGCATTGGCCGCCATAGCCGCATTGGCCGCCATAGCC
GCATTGGCGATACACTGGATCATATCAGAGCACTTGATCGCCTACAAGAA
GTTCCCCATGAGGGTCCA-3′)
SEQ ID NO: 68
(5′-CGACATAGCAGAATAGGCATTATTCAACAGAGGAGAACAAGAAATG
GAGATACACTGGATCATATCAGAGCACTTGATCGCCTACAAGAAGTTCCC
CATGAGGGTCCA-3′)
SEQ ID NO: 69
(5′-CGCCATAGCCGCATTGGCGTGACCCGCCAGCGCCGCGCGCGCAACG
GCGATACACTGGATCATATCAGAGCACTTGATCGCCTACAAGAAGTTCCC
CATGAGGGTCCA-3′)
SEQ ID NO: 70
(5′-CGCCGCCGCCGCCGCCGCCGCAGCCGCGGCCGCCGCCGCACCTATG
ATACACTGGATCATATCAGAGCACTTGATCGCCTACAAGAAGTTCCCCAT
GAGGGTCCA-3′),
SEQ ID NO: 71
(5′-GTAAAGAAGAAGAAAATCAAGAGAGAGATTAAGATTCTGGATCATA
TCAGAGCACTTGATCGCCTACAAGAAGTTCCCCATGAGGGTCCA-3′)

respectively coding the peptide of sequences SEQ ID NO: 3, 5, 56-60, 63.

The invention also relates to polynucleotides with nucleotidic sequences complementary to one of the sequence as described above and to sequences hybridizing to said polynucleotides under stringent conditions.

The invention further relates to a genetic construct consisting of or comprising a polynucleotide as defined herein, and regulatory sequences (such as a suitable promoter(s), enhancer(s), terminator(s), etc.) allowing the expression (e.g. transcription and translation) of a peptide according to the invention in a host cell.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting aspect, a genetic construct of the invention comprises i) at least one nucleic acid of the invention; operably connected to ii) one or more regulatory elements, such as a promoter and optionally a suitable terminator; and optionally also iii) one or more further elements of genetic constructs known per se; in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration.

These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used. For example, regulatory sequences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence as defined herein, e.g. a coding sequence, to which it is operably linked.

Some particularly preferred promoters include, but are not limited to, promoters known per se for the expression in the host cells mentioned herein; and in particular promoters for the expression in the bacterial cells.

A selection marker should be such that it allows, i.e. under appropriate selection conditions, host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism.

An expression marker or reporter gene should be such that—in the host cell or host organism—it allows for detection of the expression of a gene or nucleotide sequence present on the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression in bacterial cells, such as those mentioned herein. For some (further) non-limiting examples of the promoters, selection markers, leader sequences, expression markers and further elements that may be present/used in the genetic constructs of the invention, such as terminators, transcriptional and/or translational enhancers and/or integration factors, reference is made to the general handbooks such as Sambrook et al. Other examples will be clear to the skilled person.

The genetic constructs of the invention may generally be provided by suitably linking the nucleotide sequence(s) of the invention to the one or more further elements described above, for example using the techniques described in the general handbooks such as Sambrook et al.

Often, the genetic constructs of the invention will be obtained by inserting a nucleotide sequence of the invention in a suitable (expression) vector known per se.

The nucleic acids of the invention and/or the genetic constructs of the invention may be used to transform a host cell or host organism, i.e. for expression and/or production of the peptides of the invention.

Thus, in another aspect, the invention relates to a host or host cell that expresses (or that under suitable circumstances is capable of expressing) a peptide of the invention; and/or that contains a polynucleotide of the invention or genetic construct of the invention. Suitable hosts or host cells will be clear to the skilled person, and may for example be any suitable fungal, prokaryotic or eukaryotic cell or cell line or any suitable fungal, prokaryotic or eukaryotic organism, for example: a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis; a fungal cell, including but not limited to cells from species of Trichoderma, for example from Trichoderma reesei; of Neurospora, for example from Neurospora crassa; of Sordaria, for example from Sordaria macrospore; of Aspergillus, for example from Aspergillus niger or from Aspergillus sojae; or from other filamentous fungi; a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica; an amphibian cell or cell line, such as Xenopus oocytes; an insect-derived cell or cell line, such as cells/cell lines derived from lepidoptera, including but not limited to Spodoptera SF9 and Sf21 cells or cells/cell lines derived from Drosophila, such as Schneider and Kc cells; a plant or plant cell, for example in tobacco plants; and/or a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells.

Method of Preparing Peptides

The invention further relates to methods for preparing or generating the peptides of the invention.

The peptides of the invention may be produced by any well-known procedure in the art, including chemical synthesis technologies and recombinant technologies.

Examples of chemical synthesis technologies are solid phase synthesis and liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoe (t-butoxycarbonyl), Cl—Z (2-chlorobenzyloxycarbonyl), Br—Z (2-bromobenzyloyycarbonyl), Bzl (benzyl), Fmoc (9-fluorenylmcthoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Clz-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

Alternatively, the peptide may be synthesized using recombinant techniques. In this case, a nucleic acid and/or a genetic construct according to the invention as described above is/are used.

The method of producing the peptide may optionally comprise the steps of purifying said peptide, chemically modifying said peptide, and/or formulating said peptide into a pharmaceutical composition.

Pharmaceutical Compositions and Combinations

The invention further relates to a pharmaceutical composition comprising a peptide of the invention, together with a pharmaceutically acceptable carrier.

More particularly, the invention relates to a pharmaceutical composition comprising a PP2Ac peptide of the invention or a PP2Ac chimeric peptide of the invention, together with a pharmaceutically acceptable carrier.

The invention further relates to a pharmaceutical composition comprising the DPT-PP2A peptide of the invention, together with a pharmaceutically acceptable carrier.

The pharmaceutical composition as described herein may further include a second peptide, also designated as a C9x peptide, comprising or consisting of the amino acid sequence:

a) Y-X4-ETLDGI-X5-EQWA-X6-S-X7 (SEQ ID NO: 72) wherein

X4 is valine or isoleucine;

X5 is phenylalanine or leucine;

X6 is arginine or histidine;

X7 is vacant or is glutamate, or glutamate-aspartate, or glutamate-aspartate-leucine;

b) a substantially homologous peptide deriving from sequence SEQ ID NO:72 by one or more conservative substitutions; or

c) a proteolysis-resistant peptide deriving from the peptide defined in a) or b) by one or more chemical modifications.

The peptide C9x induces cell apoptosis, in vitro and/or in vivo.

In a preferred embodiment, X4 is valine; X5 is phenylalanine; and X6 is histidine.

Preferably, in the sequence SEQ ID NO: 72, the amino acids shown in bold are unmodified: Y-X4-ETLDGI-X5-EQWA-X6-S-X7.

A preferred C9x peptide is YVETLDGIFEQWAHSEDL (SEQ ID NO: 18), also designated “C9h”, or YIETLDGILEQWARSEDL (SEQ ID NO: 10), also designated “C9”.

In particular the C9x peptide is fused to a cell penetrating peptide as described herein.

In a particular preferred embodiment, the pharmaceutical compositions as described herein may include a second peptide, also designated “DPT-C9” peptide, comprising, or consisting of, the amino acid sequence:

a) X1-KKKIKREI-X2-X3-Y-X4-ETLDGI-X5-EQWA-X6-S-X7 (SEQ ID NO:6)

wherein

X1 is vacant, is a lysine residue, or valine-lysine;

X2 is vacant, is a lysine residue, or lysine-isoleucine;

X3 is vacant or is an amino acid sequence of one to 4 amino acids;

X4 is valine or isoleucine;

X5 is phenylalanine or leucine;

X6 is arginine or histidine;

X7 is vacant or is glutamate, or glutamate-aspartate, or glutamate-aspartate-leucine;

b) a substantially homologous peptide deriving from sequence SEQ ID NO:6 by one or more conservative substitutions; or

c) a proteolysis-resistant peptide deriving from the peptide defined in a) or b) by one or more chemical modifications.

The DPT-C9 peptide induces cell apoptosis, in vitro and/or in vivo.

The terms “substantially homologous”, “conservative substitution”, and the “chemical modifications” likely to confer resistance to proteolysis are as defined above.

In a preferred embodiment, X1 is valine-lysine; X2 is lysine-isoleucine; and X3 is vacant.

In another preferred embodiment, X4 is valine; X5 is phenylalanine; and X6 is histidine.

Preferably, in the sequence SEQ ID NO:6, the amino acids shown in bold are unmodified: X1-KKKIKREI-X2-X3-Y-X4-ETLDGI-X5-EQWA-X6-S-X7.

A preferred peptide is VKKKKIKREIKI-YVETLDGIFEQWAHSEDL (SEQ ID NO:7), also designated “DPT-C9h”, or VKKKKIKREIKI-YIETLDGILEQWARSEDL (SEQ ID NO:8) which were also found to induce cell apoptosis.

The pharmaceutical composition may also include any other active principle, such as in particular an anti-cancer agents, e.g. conventional cytotoxic chemotherapies with inhibitors of DNA replication such as DNA binding agents in particular alkylating or intercalating drugs, antimetabolite agents such as DNA polymerase inhibitors, or topoisomerase I or II inhibitors, or with anti-mitogenic agents such as alkaloids. These cytotoxic compounds include for instance actinomycin D, adriamycin, bleomycine, carboplatin, cisplatin, chlorambucil, cyclophosphamide, doxorubicin, etoposide, 5-fluorouracil, 6-mercaptopurine melphalan, methotrexate, paclitaxel, taxotere, vinblastine, and vincristine.

The DPT-PP2A peptide, or the PP2Ac peptide, or the PP2Ac chimeric peptide of the invention may also be provided as combination products with the DPT-C9 peptide as defined above and/or with any other active principle, for co-administration or separate administration to a patient.

Accordingly, the invention also relates to combination products which comprise a DPT-PP2A peptide, a PP2Ac peptide, or a PP2Ac chimeric peptide as defined above and a second peptide which is a DPT-C9h peptide as defined above, for simultaneous, separate or sequential use for the treatment of a cell proliferating disease.

The DPT-PP2A peptide, or the PP2Ac peptide, or the PP2Ac chimeric peptide or the pharmaceutical composition or combination products of the invention may be administered by any convenient route including intravenous, oral, transdermal, subcutaneous, mucosal, intramuscular, intrapulmonary, intranasal, parenteral, rectal, vaginal and topical. Intranasal route is of particular interest. Advantageously, intra-tumoral administration is contemplated.

In a preferred embodiment, the PP2Ac peptide may be administered by electroporation. Electroporation, also known as electropermeabilization or electroinjection, is the permeabilization of cell membranes as a consequence of the application of certain short and intense electric fields across the cell membrane, the cells or the tissues. Typically, electroporation consists of injecting compounds, preferably via intramuscular or intradermal route, followed by applying a series of electric pulses by means of electrodes connected to a generator. The conditions for applying an electric field in the injection zone are now well known to those persons skilled in the art, and are in particular described in the U.S. Pat. No. 5,468,223. Those persons skilled in the art will be able to adapt these conditions according to each case. The electric field may be 50-200 microseconds pulses of high-strength electric fields in the range of 1-5000 V/cm and with a frequency between 0.1 and 1,000 hertz. Typically, a sequence of eight 100 microseconds pulses of 1000-1500 V/cm with a frequency of 1 hertz is applied.

The peptide is formulated in association with a pharmaceutically acceptable carrier.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The dosing is selected by the skilled person so that a pro-apoptotic effect is achieved, and depends on the route of administration and the dosage form that is used. Total daily dose of DPT-PP2A peptide administered to a subject in single or divided doses may be in amounts, for example, of from about 0.001 to about 100 mg/kg body weight daily and preferably 0.01 to 10 mg/kg/day. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

Therapeutic Applications

The DPT-PP2A peptide, the PP2Ac peptide, the chimeric PP2Ac peptide as defined above, the pharmaceutical composition or combination products of the invention may be used for inducing cell apoptosis in vitro and/or in vivo. More specifically, they can find application for inhibiting cell proliferation in vitro and/or in vivo, in particular for treating hyperproliferative diseases.

The invention thus also relates to a DPT-PP2A peptide, a PP2Ac peptide or a chimeric PP2Ac peptide of the invention for use for treating a hyperproliferative disease.

The invention also provides a method of treatment of a hyperproliferative disease in a patient in need thereof, which method comprises administering said patient with a DPT-PP2A peptide, a PP2Ac peptide or a chimeric PP2Ac peptide of the invention.

The hyperproliferative disorder may be cancer, such as a haematologic cancer, in particular acute myelogenous leukaemia (AML), chronic lymphocytic leukaemia (CLL), multiple myeloma, Hodgkin's disease, non-Hodkin's lymphoma, B cell, cutaneous T cell lymphoma, or a non-haematologic cancer, for instance brain, epidermoid (in particular lung, breast, ovarian), head and neck (squamous cell), bladder, gastric, pancreatic, head, neck, renal, prostate, colorectal, oesophageal or thyroid cancer, and melanoma.

Different types of cancers may include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendothelio-sarcoma, synovioma, mesothelioma, Ewing's tumor, leiomyosarcoma, rhabdomyosarcoma, lymphoma, leukemia, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma, uveal melanoma and breast cancer.

More particularly the DPT-PP2A peptide, the PP2Ac peptide, the chimeric PP2Ac peptide, the pharmaceutical composition or combination pharmaceutical products described herein are useful in the treatment of cancers which exhibit a deregulation of PP1 and/or PP2A or which exhibit an over-expression of the anti-apoptotic protein Bcl-2, an apoptotic regulator that interacts with and is controlled by PP1 and PP2A. High levels of expression of the human bcl-2 gene have been found in all lymphomas with t (14; 18) chromosomal translocations including most follicular B cell lymphomas and many large cell non-Hodgkin's lymphomas. High levels of expression of the bcl-2 gene have also been found in leukemias that do not have a t(14; 18) chromosomal translocation, including lymphocytic leukemias of the pre-B cell type, neuroblastomas, nasophryngeal carcinomas, and many adenocarcinomas of the prostate, breast, and colon. Especially overexpression of Bcl-2 was found in chronic lymphocytic leukemia (CLL) (Deng et al, 2009 Blood. 8; 113(2):422-8; Prickett et al, 2004 J. Biol. Chem. 279, 38912-38920).

In a preferred embodiment, the cancer tumor is thus a lymphoma, especially a leukemia, such as chronic lymphocytic leukemia (CLL).

Furthermore, DPT-PP2A peptide, the PP2Ac peptide, the chimeric PP2Ac peptide, the pharmaceutical composition or combination products may be used for the treatment of metastases.

According to another embodiment, the hyperproliferative disorder may be a non-cancerous hyperproliferative disorder such as benign hyperplasia of the skin (e.g., psoriasis) or prostate (e.g., benign prostatic hypertrophy (BPH)), rheumatoid arthritis, inflammatory bowel disease, osteoarthritis, leiomyomas, adenomas, lipomas, hemangiomas, fibromas, vascular occlusion, restenosis, atherosclerosis, or oral hairy leukoplakia.

The DPT-PP2A peptide, the PP2Ac peptide, the chimeric PP2Ac peptide as defined above, the pharmaceutical composition or combination products of the invention may also be used for treating parasitic diseases.

The invention thus also relates to a DPT-PP2A peptide, a PP2Ac peptide or a chimeric PP2Ac peptide of the invention for use for treating a parasitic disease.

In particular, the DPT-PP2A peptide, a PP2Ac peptide or a chimeric PP2Ac peptide of the invention may have the ability to decrease the parasite load in a subject of at least 50%, 60%, 70%, 80%, 90% or 100%.

The invention also provides a method of treatment of a parasitic disease in a patient in need thereof, which method comprises administering said patient with a DPT-PP2A peptide, a PP2Ac peptide or a chimeric PP2Ac peptide of the invention.

Preferably, the parasitic disease is due to a parasite that belongs to the species Trypasonoma, Theileria or Plasmodium.

The parasitic disease caused by the Trypanosoma may be sleeping sickness disease in humans, Chagas disease in humans, Nagana disease in ruminant livestock, horses and pigs, Trypanosomiasis in birds, dourine or covering sickness in horses and other Equidae.

The parasitic disease caused by Theileria may be the tropical theleriosis, the Mediterranean Coast Fever, the East Coast Fever or the equine or ovine piroplasmosis.

The parasitic disease caused by Plasmodium may be malaria.

Screening Method

In an aspect, the invention also relates to a method for screening a compound that inhibits the interaction between caspase 9 and PP2A, said method comprising the step of:

a) incubating a PP2Ac peptide or a chimeric peptide as defined above and a DPT-C9h peptide as defined above in the presence or absence of said compound,

b) comparing the level of interaction between the PP2Ac and the DPT-C9h peptide in the presence or absence of said compound,

wherein a decreased level of interaction between the PP2Ac and DPT-C9h peptide in the presence of said compound in comparison to the level of interaction between the PP2Ac and DPT-C9h peptide in the absence of said compound indicates that said compound inhibits the interaction between caspase 9 and PP2A.

The screening method according to the invention may be performed by using different techniques, known by the one skilled in the art, for measuring protein-protein interactions. For example, the techniques may be:
methods based on the determination of the effect of the molecule to be tested on the formation of a complex, in an acellular medium, between the first and the second polypeptide, i.e. PP2Ac and DPT-C9h. Such a method may preferentially be performed on a solid support. Typically, one of the polypeptide forming the complex, fixed on a solid support, is contacted with the other polypeptide, labelled with an appropriate marker, in the presence of the molecule to be tested. The determination of the amount of the labelled polypeptide attached to the solid support through the interaction between the two polypeptides, allows to measure this interaction, and to evaluate the inhibiting effect of the molecule to be tested. The solid support may be, for example, a microtitration plate, which allows to evaluate several molecules in parallel.
methods based on the two-hybrid technology: in this case, the first polypeptide comprising the PP2Ac peptide is fused to the activation domain of a transcription factor, and the second polypeptide comprising the DPT-C9h peptide is fused to the DNA binding domain of the said transcription factor (or alternatively, the first polypeptide comprising the DPT-C9h peptide is fused to the activation domain of a transcription factor, and the second polypeptide comprising the PP2Ac peptide is fused to the DNA binding domain of the said transcription factor). When these polypeptide are co-expressed in a host cell containing a reporter gene under the control of a promoter recognized by the said transcription factor, expression of the reporter gene depends on the interaction between the two polypeptides, and thereby allows to detect this interaction, and to evaluate the effects of potential inhibitors.
Methods based of the BRET (Bioluminescence Resonance Energy Transfer) technology (for review, see BERTRAND et al., J Recept Signal Transduct Res, 22, 533-41, 2002). In this case, the PP2Ac peptide is expressed as a protein fused to the luciferase (PP2Ac-Luciferase) using a suitable vector, such as the vector pRLuc, whereas the DPT-C9h peptide is expressed in the form of a protein fused to the GFP2 (DPT-C9h-GFP2), using a suitable vector, such as the pGFP2 vector. Alternatively, the PP2Ac peptide is fused to the GFP2 and the DPT-C9h peptide is fused to the luciferase. Expression vectors pGFP2 and pRLuc may be obtained by Perkin Elmer. When these fusion proteins are co-expressed in a mammalian cell (e.g. HeLa, COS, 293T), the interaction between the two peptides is reflected, after excitement by adding the luciferase substrate (for example DeepBlueC, Perkin Elmer), by energy transfer to the GFP2 fusion protein and emission of a fluorescent signal that can be detected.

The invention will be illustrated in further details in the following examples, which should be regarded as illustrative and not limiting the scope of the present application.

### BRIEF DESCRIPTION OF THE SEQUENCES

SEQ ID NO: 1 corresponds to the general sequence of the DPT-PP2A peptide.

SEQ ID NO: 2 corresponds to the sequence of the cell penetrating peptide X1-KKKIKREI-X2-X3.

SEQ ID NO: 3 corresponds to the sequence of the PP2Ac peptide.

SEQ ID NO: 4 corresponds to the sequence of the DPT-PP2A peptide X1-KKKIKREI-X2-DTLDHIRALDRLQEVPHEGP.

SEQ ID NO: 5 corresponds to the sequence of the DPT-PP2Ah peptide.

SEQ ID NO: 6 corresponds to the general sequence of the DPT-C9 peptide.

SEQ ID NO: 7 corresponds to the sequence of the DPT-C9h peptide.

SEQ ID NO: 8 corresponds to the sequence of the DPT-C9 peptide VKKKKIKREIKI-YIETLDGILEQWARSEDL.

SEQ ID NO: 9 corresponds to the sequence of the DPT-sh1 peptide.

SEQ ID NO: 10 corresponds to the sequence of the C9m peptide.

SEQ ID NO: 11 corresponds to the sequence of the DPT-C9r peptide.

SEQ ID NO: 12 corresponds to the sequence of the Tat peptide YGRKKRRQRRR.

SEQ ID NO: 13 corresponds to the sequence of the DTLDHIRALDRL peptide.

SEQ ID NO: 14 corresponds to the sequence of the LDHIRALDRLQE peptide.

SEQ ID NO: 15 corresponds to the sequence of the HIRALDRLQEVP peptide.

SEQ ID NO: 16 corresponds to the sequence of the RALDRLQEVPHE peptide.

SEQ ID NO: 17 corresponds to the sequence of the LDRLQEVPHEGP peptide.

SEQ ID NO: 18 corresponds to the C9h peptide.

SEQ ID NO: 19 corresponds to the sequence of the TLDHIRALDRLQEVPHEGP peptide.

SEQ ID NO: 20 corresponds to the sequence of the LDHIRALDRLQEVPHEGP peptide.

SEQ ID NO: 21 corresponds to the sequence of the DHIRALDRLQEVPHEGP peptide.

SEQ ID NO: 22 corresponds to the sequence of the HIRALDRLQEVPHEGP peptide.

SEQ ID NO: 23 corresponds to the sequence of the DTLDHIRALDRLQEVPHEG peptide.

SEQ ID NO: 24 corresponds to the sequence of the DTLDHIRALDRLQEVPHE peptide.

SEQ ID NO: 25 corresponds to the sequence of the DTLDHIRALDRLQEVPH peptide.

SEQ ID NO: 26 corresponds to the sequence of the DTLDHIRALDRLQEVP peptide.

SEQ ID NO: 27 corresponds to the sequence of the TLDHIRALDRLQEVPHEG peptide.

SEQ ID NO: 28 corresponds to the sequence of the LDHIRALDRLQEVPHE peptide.

SEQ ID NO: 29 corresponds to the sequence of the TLDHIRALDRLQEVPHE peptide.

SEQ ID NO: 30 corresponds to the sequence of the LDHIRALDRLQEVPHEG peptide.

SEQ ID NO:31 corresponds to the sequence of the cell penetrating peptide (RQKRLI)₃.

SEQ ID NO: 32 corresponds to the sequence of the cell penetrating peptide (RHSRIG)₃.

SEQ ID NO: 33 corresponds to the sequence of the cell penetrating peptide RHSRIGIIQQRRTRNG.

SEQ ID NO: 34 corresponds to the sequence of the cell penetrating peptide RHSRIGIIQQRRTRNG.

SEQ ID NO: 35 corresponds to the sequence of the cell penetrating peptide RRRRRRRSRGRRRTY.

SEQ ID NO: 36 corresponds to the cell penetrating peptide X1-KKKIKREI-X1

SEQ ID NO: 37 corresponds to the sequence of the Tat peptide RKKRRQRRR.

SEQ ID NO: 38 corresponds to the sequence of the polyarginines peptide R₉.

SEQ ID NO: 39 corresponds to the sequence of the polyarginines peptide R₁₁.

SEQ ID NO: 40 corresponds to the sequence of the HA2-R₉ peptide.

SEQ ID NO: 41 corresponds to the sequence of the Penetratin peptide.

SEQ ID NO: 42 corresponds to the sequence of the Transportan peptide.

SEQ ID NO: 43 corresponds to the sequence of the Maurocalcine peptide.

SEQ ID NO: 44 corresponds to the sequence of the decalysine peptide.

SEQ ID NO: 45 corresponds to the sequence of the HIV-Tat derived PTD4 peptide.

SEQ ID NO: 46 corresponds to the sequence of the Hepatitis B virus Translocation Motif (PTM) peptide.

SEQ ID NO: 47 corresponds to the sequence of the mPrP₁₋₂₈ peptide.

SEQ ID NO: 48 corresponds to the sequence of the POD peptide.

SEQ ID NO: 49 corresponds to the sequence of the pVEC peptide.

SEQ ID NO: 50 corresponds to the sequence of the EB1 peptide.

SEQ ID NO: 51 corresponds to the sequence of the Rath peptide.

SEQ ID NO: 52 corresponds to the sequence of the CADY peptide.

SEQ ID NO: 53 corresponds to the sequence of the Histatin 5 peptide.

SEQ ID NO: 54 corresponds to the sequence of the Antp peptide.

SEQ ID NO: 55 corresponds to the sequence of the Cyt⁸⁶⁻¹⁰¹ peptide. SEQ ID NO: 56 corresponds to the sequence of the (RQKRLI)₃-PP2Ac peptide.

SEQ ID NO: 57 corresponds to the sequence of the (RHSRIG)₃-PP2Ac peptide.

SEQ ID NO: 58 corresponds to the sequence of the RHSRIGIIQQRRTRNG-PP2Ac peptide.

SEQ ID NO: 59 corresponds to the sequence of the RHSRIGVTRQRRARNG-PP2Ac peptide.

SEQ ID NO: 60 corresponds to the sequence of the RRRRRRRSRGRRRTY-PP2Ac peptide.

SEQ ID NO: 61 corresponds to the sequence of the X1-KKKIKREI-X2-X3-LDHIRALDRLQEVPHEGP peptide.

SEQ ID NO: 62 corresponds to the sequence of the X1-KKKIKREI-X2-LDHIRALDRLQEVPHEGP peptide.

SEQ ID NO: 63 corresponds to the sequence of the VKKKKIKREIKILDHIRALDRLQEVPHEGP peptide.

SEQ ID NO: 64 corresponds to the nucleotidic sequence coding for the PP2Ac peptide.

SEQ ID NO: 65 corresponds to the nucleotidic sequence coding for the DPT-PP2Ah peptide.

SEQ ID NO: 66 corresponds to the nucleotidic sequence coding for the (RQKRLI)₃-PP2Ac peptide.

SEQ ID NO: 67 corresponds to the nucleotidic sequence coding for the (RHSRIG)₃-PP2Ac peptide.

SEQ ID NO: 68 corresponds to the nucleotidic sequence coding for the RHSRIGIIQQRRTRNG-PP2Ac peptide.

SEQ ID NO: 69 corresponds to the nucleotidic sequence coding for the RHSRIGVTRQRRARNG-PP2Ac peptide.

SEQ ID NO: 70 corresponds to the nucleotidic sequence coding for the RRRRRRRSRGRRRTY-PP2Ac peptide.

SEQ ID NO: 71 corresponds to the nucleotidic sequence coding for the VKKKKIKREIKILDHIRALDRLQEVPHEGP peptide.

SEQ ID NO: 72 corresponds to the sequence of the C9x peptide.

### LEGENDS TO THE FIGURES

FIG. 1. Daudi cell line was cultured in RPMI 1640 medium in the presence of DPT-PP2Ah peptide (100 microM). At 12 and 24 h, we analyze the apoptotic effect of the peptide by annexine stainin.

FIG. 2 Cell isolated form xenograft models of lung cancer (LCF25), breast cancer (BC52) and uveal melanoma (KMP41) were cultured in DMEM medium in the absence (control) or in the presence of 100 microM of DPT-PP2Ah peptide. After 16 h of culture, apoptosis was estimated by annexin staining.

### EXAMPLE 1

### Identification of Murine and Human Caspase-9 Sequence Binding to PP2Ac and Characterization of Fusion Penetrating Peptides

Material and Methods

Cells

The following cells were used in this work. TS1αβ is a murine T cell line stably transfected with the α and β chains of the human IL-2 receptor that can be propagated independently in the presence of IL-2, IL-4 or IL-9 (Pitton et al, 1993 Cytokine, 5, 362-371). CTLL is a murine T cell line depending on IL-2 for proliferation. CTLL was cultured in RPMI-1640 supplemented with 5% heat-inactivated foetal calf serum, 10 mM Hepes, 2 mM glutamine and 5 ng/ml of rIL-2. Jurkat and Daudi cells were cultured in RPMI-1640 supplemented with 5% heat inactivated foetal calf serum, 10 mM Hepes and 2 mM glutamine. HeLa cells were cultured in DMEM supplemented with 10% heat-inactivated foetal calf serum, 10 mM Hepes and 2 mM glutamine.

Lymphokines, Antibodies, Kits and Reagents

Human rIL-2 was provided by Chiron (Paris, France). Anti-caspase-9 antibody was from Neo markers and anti-protein phosphatase 1 (PP1c) antibody was from Santa Cruz, Calbiochem or Transduction Laboratories. Polyclonal PP2A antibodies used in apoptotic studies were previously described (Ayllón et al, 2000). Annexin V-FITC was from (Beckman Coulter) Immunotech (Marseille, France). Peroxidase (PO)-conjugated goat anti-rabbit, -mouse, or -guinea pig Ig antibody were from Dako (Glostrup, Denmark).

Peptides

Peptides were synthesized in an automated multiple peptide synthesizer with solid phase procedure and standard Fmoc chemistry. The purity and composition of the peptides were confirmed by reverse phase HPLC and by amino acid analysis.

Pull Down Assays to Determine Interaction of Biotinylated Peptides with Intracellular Protein Targets

Biotinylated peptides were preincubated 2 h at 0-100 μM (in final concentration with lysate) at room temperature with 30 μl of streptavidin-coated immunomagnetic beads (Calbiochem, San Diego Calif.). During this time, 107 exponentially growing TS1αβ cells were first washed twice with PBS and then lysed 10 minutes on ice in 400 μl of lysis buffer (50 mM Tris pH7.4, 150 mM NaCl, 20% glycerol, 1% NP-40, 10 mM EDTA, 1 mM PhenylMethylSulfonyl Fluoride, 10 mM NaF, 1 mM orthovanadate, “complete, EDTA-free” protease inhibitor cocktail from Roche).

Lysates were clarified at 13000 g for 10 minutes at 4° C. and were incubated with biotinylated peptides associated with the streptavidin-coated immunomagnetic beads 2 h at 4° C. Biotinylated peptides were pulled down with streptavidin beads and washed two times in 700 μl of lysis buffer on ice. Bound proteins and unbound lysates were then analyzed by SDS-PAGE and Western Blotting using PP1c or PP2Ac antibodies.

Cell Death Assay

An Annexin-V-FITC conjugated kit (Roche) was used for the assessment of outer leaflet exposure of phosphatidylserine (PS) in the plasma membrane of apoptotic cells. Staining was performed according to the manufacturer's instructions. A total of 105 cells were analyzed by flow cytometry in a FACSCalibur cytofluometer (BD Biosciences). Necrotic cells were excluded by propidium iodide (PI) staining, and single annexin V positive cells were considered apoptotic. For apoptotic analysis the different peptides were used at 150 μM.

Results

DPT-C9, a Penetrating Peptide Containing the Caspase-9 (aa 401-418) PP2Ac Binding Sequence

A non cell penetrating peptide named C9 (Table 4) that contains the mouse caspase-9 (aa 401-418) PP2Ac binding domain was chemically synthesized.

Additionally, two DPT-penetrating peptides were generated in order to analyze the intracellular effects of the C9 sequence. The first penetrating peptide, named DPT-C9, contains the sequence resulting from the fusion of 12 aa residues from DPT-sh1 shuttle and C9 sequence. The second peptide, used as a negative control, contains also a bi-partite sequence resulting from the fusion of DPT-sh1 and C9 reverse sequences. Table 4 illustrates the sequences of these different peptides.

TABLE 4
Sequence of peptides

| | | | Cell |
|---|---|---|---|
| ORIGIN | ACRONYM | SEQUENCE | penetration |
| CK2a(human) | DPT-sh1 | VKKKKIKREIKI (SEQ ID NO: 9) | Yes |
| Caspase-9 | C9 | YIETLDGILEQWARSEDL (SEQ ID NO: 10) | No |
| (mouse) | DPT-C9 | VKKKKIKREIKI-YIETLDGILEQWARSEDL (SEQ ID | Yes |
| | | NO: 8) | |
| | DPT-C9r | VKKKKIKREIKI-LDESRAWQELIGDLTEIY (SEQ | Yes |
| | | ID NO: 11) | |

To determine the capacity of DPT-C9 to interact with PP2Ac, the inventors performed pull-down experiments using cellular extracts from IL-2-stimulated cells incubated with biotinylated peptides or beads alone. The shuttle, DPT-Sh1, as well as the control penetrating peptide Tat (of sequence YGRKKRRQRRR, SEQ ID NO:12) are internalized, as well as the peptides DPT-C9 and DPT-C9r. On the contrary, sequence containing the interaction site of caspase-9 with PP2Ac alone (C9) is not internalized. Both C9 and DPT-C9 were found to clearly interact with PP2Ac. In contrast, DPT-sh1 and DPT-C9r do not interact with PP2Ac. The inventors did not detect the presence of PP1c or caspase-9 associated to C9 or DPT-C9.

Effect of DPT-C9 on Apoptosis

Using flow cytometry detection of annexin V and PI, the inventors analyzed the capacity of DPT-C9 to induce apoptosis in TS1αβ cells cultured in presence of IL-2. In contrast to non penetrating C9 peptide, DPT-C9 induced a 6-fold increase of apoptosis. As expected the DPT-sh1 shuttle or DPT-C9r behaves as negative control.

Together these results indicate that DPT-C9 induces cell death in murine TS103 lymphocytes.

Effect of DPT-C9h on Apoptosis

DPT-C9h (VKKKKIKREIKI-YVETLDGIFEQWAHSEDL; SEQ ID NO:7), the homolog human C9 sequence, induced a 5-fold increase of apoptosis in Jurkat, Daudi and HeLa human cells. Interestingly, in contrast to DPT-C9, DPT-C9h is unable to cause cell death in murine TS1αβ or CTLL cell.

### EXAMPLE 2

### Identification of Human PP2Ac Sequence Binding to Human Caspase-9 and Characterization of Fusion Penetrating Peptide

Material and Methods

Cells and Reagents

Daudi is a tumoral B cell line which was propagated in RPMI-1640 (Gibco) supplemented with 10% heat-inactivated foetal calf serum (Gibco), 10 mM Hepes, 2 mM Glutamine and 50 mM 2-mercaptoethanol (2-ME).

Annexin V-FITC was from Beckman Coulter. Peroxidase (PO)-conjugated secondary antibodies were from Dako (Glostrup, Denmark).

The 12 amino acid peptides were synthesized in an automated multiple peptide synthesizer with solid phase procedure and standard Fmoc chemistry. The purity and composition of the peptides were confirmed by reverse phase HPLC and by amino acid analysis.

Caspase-9-Binding Assay on Cellulose-Bound Peptides Containing Human PP2Ac Sequences

Overlapping dodecapeptides covering the whole human PP2Ac sequence were prepared by atomated spot synthesis (Abimed, Langerfield, Germany) onto an amino-derived cellulose membrane, as described (Frank and Overwin, 1996 Meth. Mol. Biol. 66, 149-169; Gausepohl et al., 1992 Pept Res, 5, 315-320). The membrane was saturated using 3% BSA and 3% non-fat dry milk, incubated with purified human caspase-9 and after several washing steps, incubates with anti-caspase-9 antibody, followed by PO-conjugated secondary antibody. Positive spots were visualized using the ECL system.

Apoptosis Assay

Annexin V-FITC kit was used for detection of apoptosis and staining was performed according to the manufacturer's instructions. A total of 1×10⁵ cells were analyzed by flow cytometry in a FACSCalibur cytometer (BD Biosicences). Necrotic cells were excluded by PI staining and annexin V positive cells were considered apoptotic. The effect of DPT-PP2Ah was compared to untreated control cells or to DPT-C9h-treated cells. Penetrating peptides were used at 150 mM. The shuttle alone (DPT-sh1) was also used as a control.

Results

In Vitro Identification of Human PP2Ac Sequences Involved in Human Caspase-9 Interaction.

To identify peptides containing PP2Ac sequences able to binding to caspase-9, a series of 150 overlapping dodecapeptides (shift of 2 amino acids) from human PP2Ac (sequence deduced from NCBI, accession number NP_{—}00270601) were bound onto a cellulose membrane and incubated with purified caspase-9. Five peptides with overlapping sequences that bind to purified caspase-9 were identified:

```
(SEQ ID NO: 13)
DTLDHIRALDRL
(SEQ ID NO: 14)
    LDHIRALDRLQE
(SEQ ID NO: 15)
        HIRALDRLQEVP
(SEQ ID NO: 16)
            RALDRLQEVPHE
(SEQ ID NO: 17)
                LDRLQEVPHEGP.
```

Together, these results identify a new human caspase-9 binding site to the human PP2Ac: DTLDHIRALDRLQEVPHEGP (SEQ ID NO:3)

Design and Characterization of DPT-PP2Ah, a New Penetrating Peptide

We chemically synthesized a new DPT-penetrating peptide, DPT-PP2Ah in order to analyze the intracellular effect of PP2Ac sequence. This peptide contains the sequence resulting from the fusion of 12 amino acid residues from the published DPT-sh1 shuttle (VKKKKIKREIKI, SEQ ID NO:9) and PP2Ac sequence (SEQ ID NO:3).

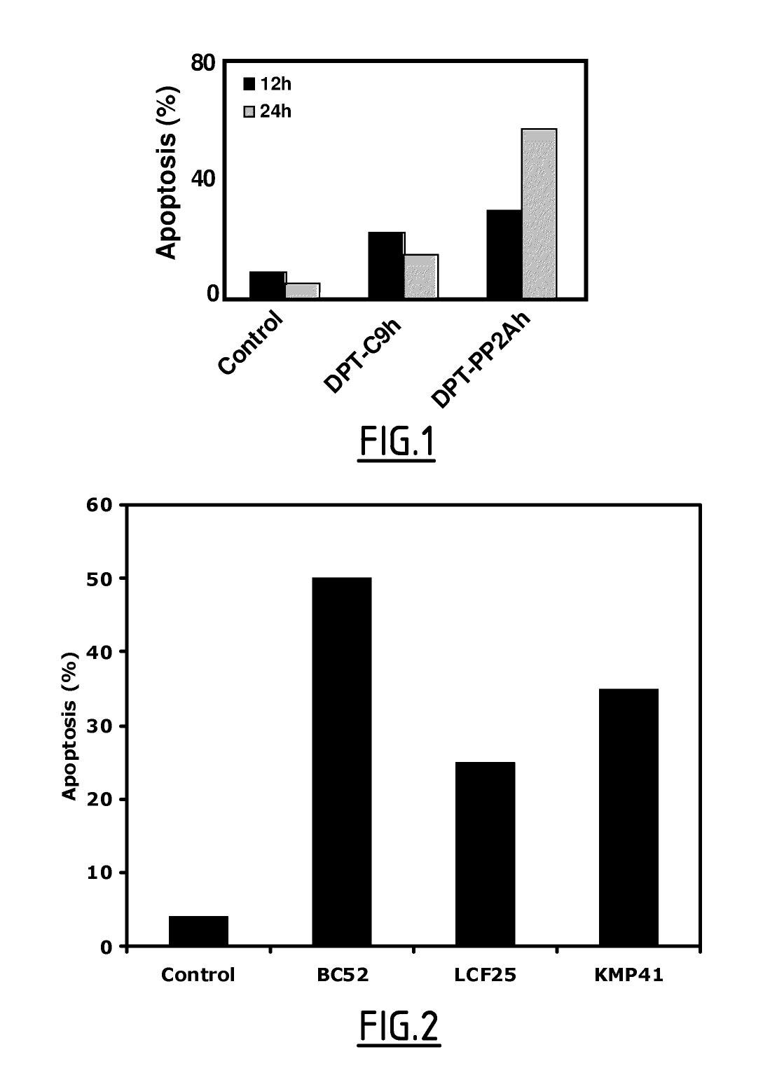

The capacity of DPT-PP2Ah to induce apoptosis was analysed in Daudi cells as well as in primary cells isolated from xenograft models of lung cancer, uveal melanoma and breast cancer. As illustrated in FIG. 1, DPT-PP2Ah strongly induces apoptosis in the human cell line Daudi compared to control non-treated. In addition, Daudi cells treated with DPT-C9h peptide show lower apoptosis than cells treated with DPT-PP2Ah penetrating peptide. FIG. 2 shows that DPT-PP2Ah also induces apoptosis in primary cells isolated from xenografts models of lung cancer (LCF25), uveal melanoma (KMP41) and breast cancer (BC52) compared to control non-treated cells.

### EXAMPLE 3

### DPT-PP2Ah Induces an In Vivo Tumor Growth Inhibition of Primary Human Breast Cancer Xenografts

Material and Methods

In Vivo Models of Primary Human Tumor Xenografts

The primary human xenografts were obtained as previously described (Marangoni et al., (2007) Clin Cancer Res 13:3989-3998, de Plater et al., (2010) Br J Cancer 103:1192-1200. Spontaneously growing mammary tumors occurring in transgenic mice were xenografted into nude immunodeficient mice to allow pharmacological assessments, and maintained from nude mouse to nude mouse serially passages.

Therapeutic Assays

For therapeutic experimental assays, 5- to 8-week old Swiss nu/nu female mice received a subcutaneous graft of tumor fragments with a volume of approximately 15 mm³. Tumors developed at the graft site 2 to 6 weeks later. Mice bearing growing tumors with a volume of 40 to 200 mm³ were individually identified and randomly assigned to the control or treatment groups (9-10 animals in each group) and treatments were started on day 1. Mice were weighed twice a week. Tumour-bearing mice were sacrificed when the tumor volume reached 2500 mm³, defined as the ethical limit. Tumor volumes and antitumor activity were evaluated as previously reported (Marangoni et al., (2007) Clin Cancer Res 13:3989-3998).

DPT-PP2Ah peptide diluted in water/glucose (1 to 25 mg/kg) was given by intraperitoneally route 7 days per week.

Results

To evaluate the potential antitumor effect of DPT-PP2Ah, we treated mice bearing the triple negative breast cancer model BC11. DPT-PP2Ah was intraperitoneally administered at 1 or 5 mg/kg, once daily for 5 weeks. At the end of the treatment, we have observed that DPT-PP2Ah induced a significant tumor growth inhibition (TGI) at the dose of 5 mg/kg. The TGI observed with the dose of 1 mg/kg was lower (FIG. 3).

1. An isolated peptide comprising:
a) the amino acid sequence DTLDHIRALDRLQEVPHEGP (SEQ ID NO: 3), or
b) an amino acid sequence substantially homologous to SEQ ID NO:3 which induces cell apoptosis; or
c) a proteolysis-resistant peptide which induces cell apoptosis and which is derived from the peptide defined in a) or b) by one or more chemical modifications.

2. The isolated peptide according to claim 1, which comprises DTLDHIRALDRLQEVPHEGP (SEQ ID NO: 3).

3. The isolated peptide according to claim 1, wherein said isolated peptide is linked to at least one cell-penetrating peptide.

4. The isolated peptide according to claim 3, wherein said at least one cell-penetrating peptide is selected from:
a)
(SEQ ID NO: 2)
a) X1-X2-KKKIKREI-X3-X4-X5-X6-X7-X8
wherein X1 is vacant, a lysine or valine residue;
X2 is vacant or a lysine residue, wherein X2 is a lysine residue when X1 is a valine residue;
X3 is vacant or a lysine residue;
X4 is vacant or an isoleucine residue when X3 is a lysine residue;
X5 is vacant or is an amino acid;
X6 is vacant or is an amino acid;
X7 is vacant or is an amino acid;
X8 is vacant or is an amino acid,
b)
(SEQ ID NO: 31)
(RQKRLI)₃,
(SEQ ID NO: 32)
(RHSRIG)₃,
(SEQ ID NO: 33)
RHSRIGIIQQRRTRNG,
(SEQ ID NO: 34)
RHSRIGVTRQRRARNG,
or
(SEQ ID NO: 35)
RRRRRRRSRGRRRTY,
c) an amino acid sequence homologous to a), and
d) Tat peptide, polyarginines peptide, HA2-R9 peptide, Penetratin peptide, Transportan peptide, Vectocell peptide, maurocalcine peptide, decalysine peptide, HIV-Tat derived PTD4 peptide, Hepatitis B virus Translocation Motif (PTM) peptide, mPrP₁₋₂₈ peptide, POD, pVEC, EB1, Rath, CADY, Histatin 5, Antp peptide, and Cyt⁸⁶⁻¹⁰¹ peptide.

5. The isolated peptide according to claim 4, wherein said peptide comprises:
a)
(SEQ ID NO: 1)
the amino acid sequence
X1-X2-KKKIKREI-X3-X4-X5-X6-X7-X8-
DTLDHIRALDRLQEVPHEGP
wherein X1 is vacant, a lysine or valine residue;
X2 is vacant or a lysine residue, wherein X2 is a lysine residue when X1 is a valine residue;
X3 is vacant or a lysine residue;
X4 is vacant or an isoleucine residue when X3 is a lysine residue;
X5 is vacant or is an amino acid;
X6 is vacant or is an amino acid;
X7 is vacant or is an amino acid;
X8 is vacant or is an amino acid;
b) an amino acid sequence at least 80% identical to SEQ ID NO:1 which induces cell apoptosis; or
c) a proteolysis-resistant peptide which induces cell apoptosis and which is derived from the peptide defined in a) or b) by one or more chemical modifications.

6. The isolated peptide according to claim 5, which comprises an amino acid sequence X1-X2-KKKIKREI-X3-X4-X5-X6-X7-X8 (SEQ ID NO:2) fused to a sequence at least 80% identical to DTLDHIRALDRLQEVPHEGP (SEQ ID NO:3) or a proteolysis-resistant peptide derived therefrom by one or more chemical modifications, wherein said proteolysis-resistant peptide induces cell apoptosis.

7. The isolated peptide according to claim 5, wherein:
(i) X5 to X8 are absent, or
(ii) X1 is valine, X2 is lysine, X3 is lysine, X4 is isoleucine and X5 to X8 are vacant.

8. The isolated peptide according to claim 5, which comprises VKKKKIKREIKIDTLDHIRALDRLQEVPHEGP (SEQ ID NO:5).

9. The isolated peptide according to claim 4, wherein said peptide comprises:
a) the amino acid sequence (RQKRLI)₃-DTLDHIRALDRLQEVPHEGP (SEQ ID NO:56), or a peptide substantially homologous thereto which induces cell apoptosis;
b) the amino acid sequence (RHSRIG)₃-DTLDHIRALDRLQEVPHEGP (SEQ ID NO:57), or a peptide substantially homologous thereto which induces cell apoptosis;
c) the amino acid sequence RHSRIGIIQQRRTRNG-DTLDHIRALDRLQEVPHEGP (SEQ ID NO:58), or a peptide substantially homologous thereto which induces cell apoptosis;
d) the amino acid sequence RHSRIGVTRQRRARNG-DTLDHIRALDRLQEVPHEGP (SEQ ID NO:59), or a peptide substantially homologous thereto which induces cell apoptosis; or
e) the amino acid sequence RRRRRRRSRGRRRTY-DTLDHIRALDRLQEVPHEGP (SEQ ID NO:60), or a peptide substantially homologous thereto which induces cell apoptosis
f) a proteolysis-resistant peptide which induces cell apoptosis and which derives from the peptide defined in a) to e) by one or more chemical modifications.

10. A polynucleotide comprising a nucleic acid encoding a peptide comprising
a) the amino acid sequence DTLDHIRALDRLQEVPHEGP (SEQ ID NO: 3), or
b) an amino acid sequence substantially homologous to SEQ ID NO:3 which induces cell apoptosis; or
c) a proteolysis-resistant peptide which induces cell apoptosis and which is derived from the peptide defined in a) or b) by one or more chemical modifications.

11. A method for producing a peptide as defined in comprising:
culturing a recombinant cell comprising a recombinant vector comprising a nucleic acid encoding a peptide comprising
a) the amino acid sequence DTLDHIRALDRLQEVPHEGP (SEQ ID NO: 3), or
b) an amino acid sequence substantially homologous to SEQ ID NO:3 which induces cell apoptosis, or
c) a proteolysis-resistant peptide which induces cell apoptosis and which is derived from the peptide defined in a) or b) by one or more chemical modifications.

12. A pharmaceutical composition comprising a peptide comprising:
a) the amino acid sequence DTLDHIRALDRLQEVPHEGP (SEQ ID NO: 3), or
b) an amino acid sequence substantially homologous to SEQ ID NO:3 which induces cell apoptosis, or
c) a proteolysis-resistant peptide which induces cell apoptosis and which is derived from the peptide defined in a) or b) by one or more chemical modifications.
together with a pharmaceutically acceptable carrier.

13. The pharmaceutical composition according to claim 12, which further comprises a second peptide comprising an amino acid sequence comprising:
a)
X1-X2-KKKIKREI-X3-X4-X5-X6-X7-X8-Y-X9-ETLDGI-X10-EQWA-X11-S—X12-X13-X14 (SEQ ID NO:6) wherein
wherein X1 is vacant, a lysine or valine residue;
X2 is vacant or a lysine residue, wherein X2 is a lysine residue when X1 is a valine residue;
X3 is vacant or a lysine residue;
X4 is vacant or an isoleucine residue when X3 is a lysine residue;
X5 is vacant or is an amino acid;
X6 is vacant or is an amino acid;
X7 is vacant or is an amino acid;
X8 is vacant or is an amino acid;
X9 is valine or isoleucine;
X10 is phenylalanine or leucine;
X11 is arginine or histidine;
X12 is vacant or is glutamate,
X13 is vacant or aspartate when X12 is glutamate,
X14 is vacant or leucine when X12 is glutamate and X13 is aspartate;
b) an amino acid sequence that is substantially homologous to SEQ ID NO: 6 and is derived from sequence SEQ ID NO:6 by one or more conservative substitutions; and
c) a proteolysis-resistant peptide derived from the peptide defined in a) or b) by one or more chemical modifications;
wherein said second peptide induces cell apoptosis.

14. The pharmaceutical composition according to claim 13, wherein in said second peptide X1 is valine, X2 is lysine, X3 is lysine, X4 is isoleucine and X5 to X8 are vacant.

15. The pharmaceutical composition according to claim 13, wherein in said second peptide X9 is valine; X10 is phenylalanine; and X11 is histidine.

16. The pharmaceutical composition according to claim 13, wherein said second peptide comprises the sequence VKKKKIKREIKI-YVETLDGIFEQWAHSEDL (SEQ ID NO:7).

17. The pharmaceutical composition according to claim 13, which further comprises another active principle.

18. A method of treating a hyperproliferative disease in a patient in need thereof comprising,
administering to said patient simultaneously, separately or sequentially a therapeutically effective amount of a combination product comprising:
a first peptide comprising an amino acid sequence selected from:
a) the amino acid sequence DTLDHIRALDRLQEVPHEGP (SEQ ID NO: 3), or
b) an amino acid sequence substantially homologous to SEQ ID NO:3 which induces cell apoptosis, and
c) a proteolysis-resistant peptide which induces cell apoptosis and which is derived from the peptide defined in a) or b) by one or more chemical modifications; and
a second peptide comprising an amino acid sequence selected from:
c)
X1-X2-KKKIKREI-X3-X4-X5-X6-X7-X8-Y-X9-ETLDGI-X10-EQWA-X11-S—X12-X13-X14 (SEQ ID NO:6) wherein
wherein X1 is vacant, a lysine or valine residue;
X2 is vacant or a lysine residue, wherein X2 is a lysine residue when X1 is a valine residue;
X3 is vacant or a lysine residue;
X4 is vacant or an isoleucine residue when X3 is a lysine residue;
X5 is vacant or is an amino acid;
X6 is vacant or is an amino acid;
X7 is vacant or is an amino acid;
X8 is vacant or is an amino acid;
X9 is valine or isoleucine;
X10 is phenylalanine or leucine;
X11 is arginine or histidine;
X12 is vacant or is glutamate.
X13 is vacant or aspartate when X12 is glutamate,
X14 is vacant or leucine when X12 is glutamate and X13 is aspartate;
d) an amino acid sequence that is substantially homologous to SEQ ID NO: 6 and is derived from sequence SEQ ID NO:6 by one or more conservative substitutions; and
e) a proteolysis-resistant peptide derived from the peptide defined in c) or d) by one or more chemical modifications.

19-20. (canceled)

21. The method of claim 20, wherein said parasitic disease is due to an infection with a parasite selected from Trypanosoma, Theileria and Plasmodium.

22. A method for screening of a compound that inhibits the interaction between caspase 9 and PP2A, said method comprising the steps of:
a) incubating a first peptide comprising:
i) the amino acid sequence DTLDHIRALDRLQEVPHEGP (SEQ ID NO: 3), or
ii) an amino acid sequence substantially homologous to SEQ ID NO:3, which induces cell apoptosis; or
iii) a proteolysis-resistant peptide which induces cell apoptosis and which is derived from the peptide defined in a) or b) by one or more chemical modifications; and
a second peptide comprising:
iii)
X1-X2-KKKIKREI-X3-X4-X5-X6-X7-X8-Y-X9-ETLDGI-X10-EQWA-X11-S—X12-X13-X14 (SEQ ID NO:6) wherein
wherein X1 is vacant, a lysine or valine residue;
X2 is vacant or a lysine residue, wherein X2 is a lysine residue when X1 is a valine residue;
X3 is vacant or a lysine residue;
X4 is vacant or an isoleucine residue when X3 is a lysine residue;
X5 is vacant or is an amino acid;
X6 is vacant or is an amino acid;
X7 is vacant or is an amino acid;
X8 is vacant or is an amino acid;
X9 is valine or isoleucine;
X10 is phenylalanine or leucine;
X11 is arginine or histidine;
X12 is vacant or is glutamate,
X13 is vacant or aspartate when X12 is glutamate,
X14 is vacant or leucine when X12 is glutamate and X13 is aspartate;
or
iv) an amino acid sequence that is substantially homologous to SEQ ID NO: 6 and is derived from sequence SEQ ID NO:6 by one or more conservative substitutions; or
v) a proteolysis-resistant peptide derived from the peptide defined in iii) or iv) by one or more chemical modifications; wherein said second peptide induces cell apoptosis in the presence and absence of said compound, and
b) comparing a level of interaction between the first peptide and the second peptide in the presence and absence of said compound,
wherein a decreased level of interaction between the first peptide and the second peptide in the presence of said compound in comparison to the level of interaction between the first peptide and the second peptide in the absence of said compound indicates that said compound inhibits the interaction between caspase 9 and PP2A.

23. The isolated peptide of claim 1, wherein said isolated peptide comprises an amino acid sequence at least 80% identical to SEQ ID NO:3.

24. The isolated peptide according to claim 9, wherein said peptide comprises:
a) a peptide at least 80% identical to the amino acid sequence (RQKRLI)₃-DTLDHIRALDRLQEVPHEGP (SEQ ID NO:56) and which induces cell apoptosis;
b) a peptide at least 80% identical to the amino acid sequence (RHSRIG)₃-DTLDHIRALDRLQEVPHEGP (SEQ ID NO:57) and which induces cell apoptosis;
c) a peptide at least 80% identical to the amino acid sequence RHSRIGIIQQRRTRNG-DTLDHIRALDRLQEVPHEGP (SEQ ID NO:58) and which induces cell apoptosis;
d) a peptide at least 80% identical to the amino acid sequence RHSRIGVTRQRRARNG-DTLDHIRALDRLQEVPHEGP (SEQ ID NO:59) and which induces cell apoptosis; or
e) a peptide at least 80% identical to the amino acid sequence RRRRRRRSRGRRRTY-DTLDHIRALDRLQEVPHEGP (SEQ ID NO:60) and which induces cell apoptosis.

25. The method of claim 11, further comprising a step of purifying the peptide.

26. The method of claim 10, wherein said peptide encoded by said nucleic acid further comprises at least one cell-penetrating peptide.

27. The method of claim 11, wherein said peptide encoded by said nucleic acid further comprises at least one cell-penetrating peptide.

28. A method of treating a hyperproliferative disease in a patient in need thereof, comprising
administering to said patient a therapeutically effective amount of a peptide of claim 1, wherein said hyperproliferative disease is
(i) a non-cancerous hyperproliferative disorder selected from the group consisting of psoriasis, benign prostatic hypertrophy, rheumatoid arthritis, inflammatory bowel disease, osteoarthritis, leiomyomas, adenomas, lipomas, hemangiomas, fibromas, vascular occlusion, restenosis, atherosclerosis, and oral hairy leukoplakia, and/or
(ii) a cancer selected from the group consisting of acute myelogenous leukemia, chronic lymphocytic leukemia, multiple myeloma, Hodgkin's disease, non-Hodgkin's lymphoma, B cell lymphoma, cutaneous T cell lymphoma, brain cancer, lung cancer, breast cancer, ovarian cancer, head and neck cancer, bladder cancer, gastric cancer, pancreatic cancer, head cancer, neck cancer, renal cancer, prostate cancer, colorectal cancer, esophageal cancer, thyroid cancer, uveal melanoma and melanoma.

29. A method of treating a parasitic disease in a patient in need thereof, comprising
administering to said patient a therapeutically effective amount of a peptide of claim 1, wherein said parasitic disease is due to an infection with a parasite selected from Trypanosoma, Theileria and Plasmodium.
